SEQUENCE LISTING (18 sequences)

SEQ ID NO: 1 - protein, 10 aa, Homo sapiens
EGPWLEEEEE

SEQ ID NO: 2 - protein, 9 aa, Homo sapiens
EGPWLEEEE

SEQ ID NO: 3 - protein, 12 aa, Homo sapiens
EGPWLEEEEEAY

SEQ ID NO: 4 - protein, 17 aa, Homo sapiens
EGPWLEEEEEAYGWMDF

SEQ ID NO: 5 - protein, 27 aa, Homo sapiens
FWVLVVVGGVLACYSLLVTVAFIIFWV

SEQ ID NO: 6 - protein, 27 aa, Homo sapiens
FWALVVVAGVLFCYGLLVTVALCVIWT

SEQ ID NO: 7 - DNA, 20 bp, Artificial Sequence
  Artificially synthesized oligonucleotide primer
gatggctgctacgtgcaact

SEQ ID NO: 8 - DNA, 19 bp, Artificial Sequence
  Artificially synthesized oligonucleotide primer
cgcaccacccgcttcttag

SEQ ID NO: 9 - DNA, 22 bp, Artificial Sequence
  Artificially synthesized oligonucleotide primer
tgcggactacaagcgaatcacg

SEQ ID NO: 10 - DNA, 22 bp, Artificial Sequence
  Artificially synthesized oligonucleotide primer
ctcagcttctggataaccctcg

SEQ ID NO: 11 - DNA, 465 bp, Homo sapiens
  CDS (63)..(368)
agagacctgagaggcaccaggcccagccgtggcaccacacacctcccagctctgcagacgagatgcagcgactgtgtgtgtatgtgctgatctttgcactggctctggccgccttctctgaagcttcttggaagccccgctcccagcagccagatgcacccttaggtacaggggccaacagggacctggagctaccctggctggagcagcagggcccagcctctcatcatcgaaggcagctgggaccccagggtcccccacacctcgtggcagacccgtccaagaagcagggaccatggctggaggaagaagaagaagcctatggatggatggacttcggccgccgcagtgctgaggatgagaactaacaatcctagaaccaagcttcagagcctagccacctcccaccccactccagccctgtcccctgaaaaactgatcaaaaataaactagtttccagtgga

SEQ ID NO: 12 - protein, 101 aa, Homo sapiens
MQRLCVYVLIFALALAAFSEASWKPRSQQPDAPLGTGANRDLELPWLEQQGPASHHRRQLGPQGPPHLVADPSKKQGPWLEEEEEAYGWMDFGRRSAEDEN

SEQ ID NO: 13 - protein, 21 aa, Homo sapiens
PTKAPDVFPIISGCRHPKDNS

SEQ ID NO: 14 - protein, 98 aa, Homo sapiens
LDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 15 - protein, 95 aa, Mus musculus
LDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 16 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - protein, 113 aa, Mus musculus
RAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 18 - protein, 21 aa, Homo sapiens
MALPVTALLLPLALLLHAARP

(19) United States
(12) Patent Application Publication
(10) Pub. No.: US 2023/0398196 A1
(43) Pub. Date: Dec. 14, 2023

(54) COMPOSITIONS AND METHODS FOR ENHANCING T CELL PENETRATION OF TUMORS AND CANCERS

(71) Applicants: Cancer Advances Inc., Durham, NC (US); Georgetown University, Washington, DC (US)

(72) Inventors: Allen Cato, Durham, NC (US); Lynda Sutton, Durham, NC (US); Jill P. Smith, Camp Hill, PA (US)

(73) Assignees: Cancer Advances Inc., Durham, NC (US); Georgetown University, Washington, DC (US)

(21) Appl. No.: 18/035,908

(22) PCT Filed: Nov. 8, 2021
(86) PCT No.: PCT/US2021/058447

§ 371 Date: May 8, 2023

Related U.S. Application Data
(60) Provisional application No. 63/110,905, filed on Nov. 6, 2020.

(51) Int. Cl. A61K 39/00; A61P 35/00; C07K 16/28; A61P 35/04

(52) U.S. Cl. CPC A61K 39/001144; A61P 35/00; A61K 39/4611; C07K 16/2818; A61P 35/04; A61K 2039/627; A61K 2039/6037; A61K 2039/505

(57) ABSTRACT

Provided are methods for enhancing anti-tumor and/or anti-cancer immunotherapies. In some embodiments, the methods include administering to a subject in need thereof a composition that includes a conjugate that includes a gastrin immunogen conjugated to an immunogenic carrier, optionally conjugated via a linker, in an amount and via a route sufficient to enhance entry into the tumor and/or cancer of an anti-tumor T cell, whereby an anti- and/or anti-cancer tumor immunotherapy is enhanced. Also provided are pharmaceutical compositions that have the conjugates for use in treating tumors and cancers, methods for treating tumors and/or cancers, and methods for sensitizing solid tumors and/or cancers in subjects to chimeric antigen receptor-T (CAR-T) cell therapies.

### CROSS-REFERENCE TO RELATED APPLICATION

This application claims the benefit of U.S. Provisional Patent Application Ser. No. 63/110,905, filed Nov. 6, 2020, the disclosure of which is incorporated herein by reference in its entirety.

### TECHNICAL FIELD

The presently disclosed subject matter relates to compositions and methods for enhancing penetration of tumors, particularly solid tumors, by immunotherapeutic molecules. In some embodiments, the presently disclosed subject matter relates to administering to a subject in need thereof an inducer of a humoral and/or cellular immune response against a gastrin peptide, wherein the inducer of a humoral and/or cellular immune response against the gastrin peptide results in the tumor becoming more accessible to immunotherapeutic molecules including but not limited to chimeric antigen receptors (CARs) and/or T cells that have been engineered to express the CARs (referred to herein as “CAR-T cells”).

### REFERENCE TO SEQUENCE LISTING

The Sequence Listing associated with the instant disclosure has been electronically submitted to the United States Patent and Trademark Office as a 5 kilobyte ASCII text file created on Nov. 5, 2021 and entitled “1734 10 25 PCT ST25.txt”. The Sequence Listing submitted via EFS-Web is hereby incorporated by reference in its entirety.

### BACKGROUND

Gastric adenocarcinoma (gastric cancer) is a common malignancy and is the world's second leading cause of cancer mortality worldwide. Novel therapeutic targets are desperately needed because the meager improvement in the cure rate of about 10% realized by adjunctive treatments to surgery is unacceptable as >50% patients with localized gastric cancer succumb to their disease. The prognosis of those with advanced gastric cancer is poor with a five-year survival of only 20-30%. The current standard of care for advanced gastric cancer in the first line setting remains a combination of a fluoropyrimidine (e.g., 5-fluorouracil; 5FU) and a platinum (e.g., cis-platinum) containing chemotherapeutic agent. Targeted therapy may offer new possibilities for the treatment of gastric cancer. Since HER2 receptors are found in approximately 20% of gastric cancers, the addition of a HER2 receptor antibody to standard chemotherapy may be beneficial as demonstrated in the ToGA study where Trastuzumab (Herceptin) was beneficial in subjects with HER2-positive gastric cancer. The Cancer Genome Atlas (TCGA) Research Network described four groups of gastric cancer based upon molecular classifications including: EBV (Epstein-Barr virus), MSI (microsatellite instability), GS (genomically stable), and CIN (chromosomal instability). The immune response to the tumor could play an important role within the EBV and MSI subgroups. With the recent use of immune checkpoint antibodies, investigators have been exploring whether this immunotherapy would be beneficial for gastric cancer. The KEYNOTE-012 study tested 39 subjects in a Phase 1 trial that were PD-L1 positive with pembrolizumab and found an overall survival of 11.4 months. The KEYNOTE-059 trial showed that pembrolizumab monotherapy was effective treating those with previously treated gastric or gastroesophageal cancer. Another PD-1 antibody, nivolumab, has been approved for first line therapy in gastric cancer in combination with chemotherapy after the results of the CheckMate-649 clinical trial. A number of clinical trials have been conducted now with various immune checkpoint antibodies and although these agents have provided additional therapeutic options for those with gastric cancer, unfortunately the median overall survival still remains less than 12 months. For these reasons novel strategies are needed to improve response of those with gastric cancer to immunotherapy. One possible reason for the still low response to immune checkpoint antibodies may be related to the paucity of tumor infiltrating CD8⁺ lymphocytes in the tumor. Another possible reason for the low response rate may be due to the fibrosis of the tumor microenvironment that prevents penetration of therapies and immune cells. Therapeutic agents that target cancer cell receptors such as HER25 have been shown to improve survival and yet most chemotherapy agents used in gastric cancer are not target-specific.

The gastrointestinal (GI) peptide gastrin is responsible for gastric acid secretion and growth of the GI tract, and gastrin mediates its effects through the cholecystokinin-B receptor or CCK-BR. Unlike the physiologic expression of gastrin in the G cells of the stomach antrum, the gastrin gene also becomes overexpressed de novo in non-endocrine epithelial cells of gastric cancer where it can stimulate growth in an autocrine fashion. Likewise, the CCK-BR also becomes over-expressed in cancer cells and this receptor is responsive to both paracrine and autocrine stimulation by gastrin. Investigators have studied the expression of gastrin and the CCK-BR from resected human gastric cancers and found that most expressed CCK-BRs and gastrin. Gastrin may also stimulate growth of gastric cancer when blood gastrin levels are increased from chronic use of high dose proton pump inhibits (PPIs), achlorhydria or Helicobacter pylori infection. Since gastrin has been shown to stimulate growth of human gastric cancer, researchers have been studying means to block gastrin's actions in gastric cancer using CCK-BR antagonists and their use in human trials.

Polyclonal Antibody Stimulator (PAS) is a therapeutic immunogen cancer vaccine comprised of a nine amino acid epitope derived from the amino-terminal sequence of gastrin-17 that is conjugated to diphtheria toxoid. PAS exerts an immunomodulatory effect by activating both B-28-30 and T-cells. PAS stimulates the production of antibodies to different epitopes of the G17 and precursor G17-Gly gastrin peptides. These antibodies can bind to gastrin peptides to prevent their interaction with the CCK-BRs on the surface of tumor cells. Preclinical studies were performed in several animal models that have CCK-BRs including gastric cancer. In animal models, PAS-generated anti-gastrin antibodies have been shown to reduce the growth and metastases. Passive immunization with PAS antibodies raised in rabbits improved survival of SCID mice bearing gastric cancers compared to diluent treated controls.

To date 22 clinical studies have been conducted with PAS. Of these, 840 patients have been enrolled in 5 clinical trials for the treatment of pancreatic cancer; 234 subjects enrolled in 5 clinic studies in gastric cancer; and 475 subjects enrolled in ten clinical studies with advanced colon cancer. In the gastric cancer clinical trial (designated GC4 Study), the median survival of those with advanced gastric cancer treated with PAS plus cisplatin and was significantly prolonged (10.8 months) in subjects that mounted a protective antibody titer against gastrin compared to subjects treated with PAS plus cisplatin and 5FU that failed to generate an antibody response (4.8 months). The only notable PAS-related adverse events in all 22 studies were injection-site reaction and pyrexia.

### SUMMARY

This Summary lists several embodiments of the presently disclosed subject matter, and in many cases, lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter relates to methods for enhancing anti-tumor and/or anti-cancer immunotherapies. In some embodiments, the methods comprise, consist essentially of, or consist of administering to a tumor and/or a cancer a composition comprising, consisting essentially of, or consisting of a conjugate comprising, consisting essentially of, or consisting of a gastrin immunogen conjugated to an immunogenic carrier, optionally conjugated via a linker, in an amount and via a route sufficient to enhance entry into the tumor and/or cancer of an anti-tumor immunotherapeutic molecule, optionally a CAR or CAR-T cell, whereby an anti- and/or anti-cancer tumor immunotherapy is enhanced. In some embodiments, the tumor and/or the cancer is a gastrointestinal tumor and/or cancer, optionally a gastrin-dependent gastrointestinal tumor and/or cancer, further optionally a pancreatic tumor and/or cancer. In some embodiments, the tumor and/or the cancer is a solid tumor and/or cancer of the gastrointestinal tract, optionally a solid tumor and/or cancer of the pancreas. In some embodiments, the tumor and/or the cancer is a gastrin-responsive cancer, such as but not limited to a gastrinoma, lung cancer, and/or thyroid cancer. In some embodiments, the immunogenic carrier is selected from the group consisting of diphtheria toxoid, tetanus toxoid, keyhole limpet hemocyanin, and bovine serum albumin. In some embodiments, the linker comprises a ε-maleimido caproic acid N-hydroxysuccinamide ester. In some embodiments, the linker and the gastrin peptide are separated by an amino acid spacer, optionally wherein the amino acid spacer is between 1 and 10 amino acids in length, further optionally wherein the amino acid spacer is 7 amino acids in length. In some embodiments, the composition further comprises an adjuvant, optionally an oil-based adjuvant. In some embodiments, the gastrin peptide comprises, consists essentially of, or consists of an amino acid sequence selected from the group consisting of EGPWLEEEEE (SEQ ID NO: 1), EGPWLEEEE (SEQ ID NO: 2), EGPWLEEEEEAY (SEQ ID NO: 3), and EGPWLEEEEEAYGWMDF (SEQ ID NO: 4).

In some embodiments, the presently disclosed subject matter also relates to pharmaceutical compositions for use in producing medicaments for treating a gastrin-associated tumors and/or cancers. In some embodiments, the pharmaceutical compositions comprise, consist essentially of, or consist of one or more conjugates comprising a gastrin immunogen in an amount sufficient to enhance anti-tumor and/or anti-cancer T cell entry into the gastrin-associated tumor and/or cancer.

In some embodiments, the presently disclosed subject matter also relates to pharmaceutical compositions for use in treating a tumor and/or a cancer, optionally, a gastrin-associated tumor and/or cancer, the pharmaceutical compositions comprising, consisting essentially of, or consisting of a conjugate comprising a gastrin immunogen in an amount sufficient to enhance anti-tumor and/or anti-cancer T cell entry into the tumor and/or cancer.

In some embodiments, the presently disclosed subject matter also relates to methods for treating tumors and/or cancers, optionally gastrin-associated tumors and/or cancers, in a subject. In some embodiments, the methods comprise administering to the subject a first composition comprising, consisting essentially of, or consisting of a conjugate comprising, consisting essentially of, or consisting of a gastrin immunogen conjugated to an immunogenic carrier, optionally conjugated via a linker, in an amount and via a route sufficient to enhance anti-tumor and/or anti-cancer T cell entry into the tumor and/or cancer; and administering to the subject a second compositions comprising an anti-tumor and/or anti-cancer T cell, wherein the anti-tumor and/or anti-cancer T cell optionally comprises a chimeric antigen receptor (CAR) that binds to a tumor-associated and/or cancer-associated antigen present on the tumor and/or the cancer, whereby the tumor and/or the cancer is treated. In some embodiments, the tumor and/or the cancer is a gastrointestinal tumor and/or cancer, optionally a gastrin-dependent gastrointestinal tumor and/or cancer, further optionally a pancreatic tumor and/or cancer. In some embodiments, the immunogenic carrier is selected from the group consisting of diphtheria toxoid, tetanus toxoid, keyhole limpet hemocyanin, and bovine serum albumin. In some embodiments, the linker comprises a ε-maleimido caproic acid N-hydroxysuccinamide ester. In some embodiments, the linker and the gastrin peptide are separated by an amino acid spacer, optionally wherein the amino acid spacer is between 1 and 10 amino acids in length, further optionally wherein the amino acid spacer is 7 amino acids in length. In some embodiments, the composition further comprises an adjuvant, optionally an oil-based adjuvant. In some embodiments, the gastrin peptide comprises, consists essentially of, or consists of an amino acid sequence selected from the group consisting of EGPWLEEEEE (SEQ ID NO: 1), EGPWLEEEE (SEQ ID NO: 2), EGPWLEEEEEAY (SEQ ID NO: 3), and EGPWLEEEEEAYGWMDF (SEQ ID NO: 4). In some embodiments, the CAR binds to an antigen selected from the group consisting of a claudin18.2 antigen, a glypican3 antigen, a mesothelin antigen, a carcinoembryonic antigen, a prostate stem cell antigen, and a CD70 antigen.

In some embodiments, the presently disclosed subject matter also relates to methods for sensitizing solid tumors and/or cancers in subjects to chimeric antigen receptor-T (CAR-T) cell therapies. In some embodiments, the methods comprise administering to a subject a first composition comprising, consisting essentially of, or consisting of a conjugate comprising, consisting essentially of, or consisting of a gastrin immunogen conjugated to an immunogenic carrier, optionally conjugated via a linker, in an amount and via a route sufficient to enhance entry of the CAR-T cell into the solid tumor and/or cancer; and administering to the subject a second composition comprising, consisting essentially of, or consisting of a CAR-T cell that is targeted against an antigen present within the solid tumor and/or cancer, whereby the solid tumor and/or cancer in the subject is sensitized to the CAR-T cell therapy. In some embodiments, the first composition and the second compositions are administered at the same time. In some embodiments, the first composition and the second compositions are administered at different times. In some embodiments, the first composition and the second compositions are administered at multiple times each. In some embodiments, the solid tumor and/or the cancer is a solid gastrointestinal tumor and/or cancer, optionally a solid gastrin-dependent gastrointestinal tumor and/or cancer, further optionally a solid pancreatic tumor and/or cancer. In some embodiments, the solid tumor and/or the cancer is a solid gastrin-responsive cancer, such as but not limited to a gastrinoma, lung cancer, and/or thyroid cancer.

In some embodiments of the presently disclosed subject matter methods, the immunogenic carrier is selected from the group consisting of diphtheria toxoid, tetanus toxoid, keyhole limpet hemocyanin, and bovine serum albumin. In some embodiments, the linker comprises a ε-maleimido caproic acid N-hydroxysuccinamide ester. In some embodiments, the linker and the gastrin peptide are separated by an amino acid spacer, optionally wherein the amino acid spacer is between 1 and 10 amino acids in length, further optionally wherein the amino acid spacer is 7 amino acids in length. In some embodiments, the first composition further comprises an adjuvant, optionally an oil-based adjuvant. In some embodiments, the gastrin peptide comprises, consists essentially of, or consists of an amino acid sequence selected from the group consisting of EGPWLEEEEE (SEQ ID NO: 1), EGPWLEEEE (SEQ ID NO: 2), EGPWLEEEEEAY (SEQ ID NO: 3), and EGPWLEEEEEAYGWMDF (SEQ ID NO: 4).

In some embodiments, the presently disclosed methods further comprise administering to the subject one or more additional anti-tumor and/or anti-cancer therapies. In some embodiments, the one or more additional anti-tumor and/or anti-cancer therapies comprises, consists essentially of, or consists of administering to the subject an immune checkpoint inhibitor. In some embodiments, the immune checkpoint inhibitor inhibits a biological activity of a target polypeptide selected from the group consisting of cytotoxic T-lymphocyte antigen 4 (CTLA4), programmed cell death-1 receptor (PD-1), and programmed cell death 1 receptor ligand (PD-L1). In some embodiments, the immune checkpoint inhibitor is selected from the group consisting of Ipilimumab, Tremelimumab, Nivolumab, Pidilizumab, Pembrolizumab, AMPS 14, AUNP12, BMS-936559/MDX-1105, Atezolizumab, MPDL3280A, RG7446, R05541267, MEDI4736, and Avelumab.

In some embodiments, the presently disclosed methods reduce and/or inhibit growth of the tumor and/or the cancer in the subject. In some embodiments, the first composition is administered in a dose selected from the group consisting of about 50 μg to about 1000 about 50 μg to about 500 about 100 μg to about 1000 about 200 μg to about 1000 and about 250 μg to about 500 μg, and optionally wherein the dose is repeated once, twice, or three times, optionally wherein the second dose is administered 1 week after the first dose and the third dose, if administered, is administered 1 or 2 weeks after the second dose. In some embodiments, the one or more additional anti-tumor and/or anti-cancer therapies is administered subsequent to the administration of at least the first dose of the composition. In some embodiments, the CAR binds to an antigen selected from the group consisting of a claudin18.2 antigen, a glypican3 antigen, a mesothelin antigen, a carcinoembryonic antigen (CEA), a prostate stem cell antigen (PSCA), and a CD70 antigen. In some embodiments, the tumor and/or the cancer is a gastric tumor and/or cancer, a pancreatic tumor and/or cancer, a liver cancer, a metastatic lesion derived therefrom, or a metastasis to the stomach, pancreas, liver, lung, brain, or any other tissue or organ of a subject.

Thus, it is an object of the presently disclosed subject matter to provide compositions and methods for methods for enhancing anti-tumor and/or anti-cancer immunotherapies.

An object of the presently disclosed subject matter having been stated hereinabove, and which is achieved in whole or in part by the compositions and methods disclosed herein, other objects will become evident as the description proceeds when taken in connection with the accompanying Figures as best described herein below.

### BRIEF DESCRIPTION OF THE FIGURES

FIGS. 1A-1F. Characterization of murine gastric cancer cells in vitro. (FIG. 1A) mRNA expression of CCK-BR is increased greater than 60-fold in YTN and NCC gastric cancer cells compared to normal mouse tissue. (FIG. 1B) mRNA expression by qRT-PCR of PD-L1 is markedly increased in in gastric cancer YTN and NCC cells compared to normal tissues. (FIG. 1C) Exogenous gastrin stimulates growth of murine NCC gastric cancer cells in vitro (p=0.004). (FIG. 1D) Gastrin peptide expression is detected in NCC gastric cancer cells. (FIG. 1E) Gastrin peptide expression is detected in YTN gastric cancer cells. (FIG. 1F) Control cells stained with the secondary antibody only show no evidence of non-specific immunoreactivity. Scale bar 200 μm.

FIGS. 2A-2G. PAS vaccination alone or in combination with PD-1 Ab inhibits growth and metastases of YTN gastric cancer tumors in mice. (FIG. 2A) YTN tumor volumes over time for each treatment group and respective slope of the line are shown. PD-1Ab monotherapy did not alter rate of YTN tumor growth compared to PB S-treated controls. Tumors of mice treated with PAS monotherapy (p=0.023) or in combination with PD-1 Ab (p=0.0003) significantly reduced tumor growth in mice. (FIG. 2B) Final tumor mass ex-vivo showed a reduction in size in mice treated with PAS in combination with the PD-1 Ab (p=0.09). (FIG. 2C) Number of metastases for each treatment group demonstrates that metastases were only observed in Control (PBS-treated) mice and in mice treated with PD-1 Ab. No metastases were found in mice treated with PAS monotherapy or PAS in combination with the PD-1 Ab. (FIGS. 2D-2G) Metastases were confirmed histologically by H&E stain. (FIG. 2D) Invasive YTN tumor invading the stomach wall. (FIG. 2E) Peritoneal seeding with metastases. (FIG. 2F) Invasion of YTN tumor cells in the mesentery fat. (FIG. 2G) YTN cancer invading the abdominal wall skeletal muscle.

FIGS. 3A-3D. Effects of PAS and PD-1 Ab treatment on tumor proliferation and fibrosis. (FIG. 3A) The mean number±SEM of Ki67 stained cells is shown for each cohort of YTN tumors. Ki67 immunoreactivity in PD-1 Ab tumors increased compared to PBS-treated controls (p<0.05). Ki67 staining was significantly reduced in tumors of mice treated with PAS monotherapy or in combination with the PD-1 Ab (p<0.0001). (FIG. 3B) Representative images from tumors reacted with Ki67 antibody for each treatment group is show at low magnification (2×, bar scale, 2 mm) and at a higher magnification (40×, Box insert). (FIG. 3C) Representative images of tumors from each treatment group stained for fibrosis with Masson's trichrome stain (scale bar=200 μm). (FIG. 3D) Mean values±SEM for fibrosis staining is shown for each treatment as analyzed by integrative density. Intratumoral fibrosis was decreased in all treatment groups compared to PBS-treated control tumors. Tumors of the combination therapy group also exhibited less fibrosis than tumors of the mice treated with PD-1 Ab monotherapy. (Compared to PBS **p<0.01; ***p<0.001; compared to PD-1 Ab #p<0.05).

FIGS. 4A-4D. PAS monotherapy and in combination with PD-1 Ab alter the tumor immune cell signature. (FIG. 4A) Representative low magnification tumor from each treatment or control group (scale bar 600 μm) and a higher magnification (20×; Box insert) of tumors stained with and antibody for CD8⁺ T-lymphocytes. (FIG. 4B) Columns represent the mean±SEM of the number of CD8⁺ immunoreactive cells in sections of YTN tumors from each group. PAS monotherapy and in combination with a PD-1 Ab significantly increase the number of CD8− immunoreactive T-cells in the YTN tumors compared to tumors of PBS-treated mice. The combination of PAS with PD-1 Ab also markedly increased the number of CD8⁺ cells compared to PAS monotherapy. (FIG. 4C) Representative low magnification tumor from each treatment or control group (scale bar 600 μm) and a higher magnification (20×; Box insert) of tumors stained with and antibody for arginase to detect M2-polarized tumor-associated macrophages (TAMs). (FIG. 4D) Computer analysis with integrative density of the images confirmed that the immunoreactivity was significantly decreased in tumors of PAS-treated mice. Tumors of mice treated with both PAS and the PD-1 Ab had even further decreased immunoreactivity of arginase positive TAMs. ***p<0.001 compared to PBS; ^{###}p<0.001, compared to PAS.

FIGS. 5A-51I. CCK-BR protein expression by immunohistochemistry in human gastric cancer and normal tissues from a human gastric tissue array (US Biomax #BC01011). A stomach carcinoma (multi-tissue combined panel) tissue array (Catalog No. BC01011; US Biomax, Inc., Rockville, Maryland, United States of America) was stained with a CCK-BR antibody (Catalog No. 77077; Abcam, Waltham, Massachusetts, United States of America) at a titer of 1:200 overnight at 4° C. (FIGS. 5A-5C) Gastric cancer images representative of the intestinal type histology are shown. (FIGS. 5D and 5E) Representative images of gastric cancers with the diffuse histologic type are shown. (FIG. 5F) Gastric carcinoma mucinous adenocarcinoma. (FIG. 5G) Gastric cancer signet ring histology; arrows point to signet ring cells. (FIG. 5H) Histology normal human stomach.

### DETAILED DESCRIPTION

Headings are included herein for reference and to aid in locating certain sections. These headings are not intended to limit the scope of the concepts described therein under, and these concepts can have applicability in other sections throughout the entire description.

### I. Definitions

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the presently disclosed and claimed subject matter.

Following long-standing patent law convention, the terms “a”, “an”, and “the” refer to “one or more” when used in this application, including in the claims. For example, the phrase “an inhibitor” refers to one or more inhibitors, including a plurality of the same inhibitor. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to whole number values between 1 and 100 and greater than 100.

Unless otherwise indicated, all numbers expressing quantities of ingredients, reaction conditions, and so forth used in the specification and claims are to be understood as being modified in all instances by the term “about”. The term “about”, as used herein when referring to a measurable value such as an amount of mass, weight, time, volume, concentration, or percentage, is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods and/or employ the disclosed compositions. Accordingly, unless indicated to the contrary, the numerical parameters set forth in this specification and attached claims are approximations that can vary depending upon the desired properties sought to be obtained by the presently disclosed subject matter.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D.

As used herein, the terms “antibody” and “antibodies” refer to proteins comprising one or more polypeptides substantially encoded by immunoglobulin genes or fragments of immunoglobulin genes. Immunoglobulin genes typically include the kappa (κ), lambda (λ), alpha (α), gamma (γ), delta (δ), epsilon (ε), and mu (μ) constant region genes, as well as myriad immunoglobulin variable region genes. Light chains are classified as either κ or λ. In mammals, heavy chains are classified as γ, μ, α, δ, or ε, which in turn define the immunoglobulin classes, IgG, IgM, IgA, IgD, and IgE, respectively. Other species have other light and heavy chain genes (e.g., certain avians produced what is referred to as IgY, which is an immunoglobulin type that hens deposit in the yolks of their eggs), which are similarly encompassed by the presently disclosed subject matter. In some embodiments, the term “antibody” refers to an antibody that binds specifically to an epitope that is present on a gastrin gene product, including but not limited to an epitope that is present within an amino acid sequence as set forth in EGPWLEEEEE (SEQ ID NO: 1), EGPWLEEEE (SEQ ID NO: 2), EGPWLEEEEEAY (SEQ ID NO: 3), and EGPWLEEEEEAYGWMDF (SEQ ID NO: 4).

A typical immunoglobulin (antibody) structural unit is known to comprise a tetramer. Each tetramer is composed of two identical pairs of polypeptide chains, each pair having one “light” chain (average molecular weight of about 25 kilodalton (kDa)) and one “heavy” chain (average molecular weight of about 50-70 kDa). The two identical pairs of polypeptide chains are held together in dimeric form by disulfide bonds that are present within the heavy chain region. The N-terminus of each chain defines a variable region of about 100 to 110 or more amino acids primarily responsible for antigen recognition. The terms variable light chain (V_{L}) and variable heavy chain (V_{H}) refer to these light and heavy chains, respectively.

Antibodies typically exist as intact immunoglobulins or as a number of well-characterized fragments that can be produced by digestion with various peptidases. For example, digestion of an antibody molecule with papain cleaves the antibody at a position N-terminal to the disulfide bonds. This produces three fragments: two identical “Fab” fragments, which have a light chain and the N-terminus of the heavy chain, and an “Fc” fragment that includes the C-terminus of the heavy chains held together by the disulfide bonds. Pepsin, on the other hand, digests an antibody C-terminal to the disulfide bond in the hinge region to produce a fragment known as the “F(ab)′₂” fragment, which is a dimer of the Fab fragments joined by the disulfide bond. The F(ab)′₂ fragment can be reduced under mild conditions to break the disulfide linkage in the hinge region, thereby converting the F(ab′)₂ dimer into two Fab′ monomers. The Fab′ monomer is essentially an Fab fragment with part of the hinge region (see e.g., Paul, 1993 for a more detailed description of other antibody fragments). With respect to these various fragments, Fab, F(ab′)₂, and Fab′ fragments include at least one intact antigen binding domain, and thus are capable of binding to antigens.

While various antibody fragments are defined in terms of the digestion of an intact antibody, one of skill will appreciate that various of these fragments (including, but not limited to Fab′ fragments) can be synthesized de novo either chemically or by utilizing recombinant DNA methodology. Thus, the term “antibody” as used herein also includes antibody fragments either produced by the modification of whole antibodies or synthesized de novo using recombinant DNA methodologies. In some embodiments, the term “antibody” comprises a fragment that has at least one antigen binding domain.

Antibodies can be polyclonal or monoclonal. As used herein, the term “polyclonal” refers to antibodies that are derived from different antibody-producing cells (e.g., B cells) that are present together in a given collection of antibodies. Exemplary polyclonal antibodies include but are not limited to those antibodies that bind to a particular antigen and that are found in the blood of an animal after that animal has produced an immune response against the antigen. However, it is understood that a polyclonal preparation of antibodies can also be prepared artificially by mixing at least non-identical two antibodies. Thus, polyclonal antibodies typically include different antibodies that are directed against (i.e., binds to) different epitopes (sometimes referred to as an “antigenic determinant” or just “determinant”) of any given antigen.

As used herein, the term “monoclonal” refers to a single antibody species and/or a substantially homogeneous population of a single antibody species. Stated another way, “monoclonal” refers to individual antibodies or populations of individual antibodies in which the antibodies are identical in specificity and affinity except for possible naturally occurring mutations, or post-translational modifications that can be present in minor amounts. Typically, a monoclonal antibody (mAb) is generated by a single B cell or a progeny cell thereof (although the presently disclosed subject matter also encompasses “monoclonal” antibodies that are produced by molecular biological techniques as described herein). Monoclonal antibodies (mAbs) are highly specific, typically being directed against a single antigenic site. Furthermore, in contrast to polyclonal antibody preparations, a given mAb is typically directed against a single epitope on the antigen.

In addition to their specificity, mAbs can be advantageous for some purposes in that they can be synthesized uncontaminated by other antibodies. The modifier “monoclonal” is not to be construed as requiring production of the antibody by any particular method, however. For example, in some embodiments, the mAbs of the presently disclosed subject matter are prepared using the hybridoma methodology first described by Kohler et al., 1975, and in some embodiments, are made using recombinant DNA methods in bacterial or eukaryotic animal or plant cells (see e.g., U.S. Pat. No. 4,816,567, the entire contents of which are incorporated herein by reference). mAbs can also be isolated from phage antibody libraries using the techniques described in Clackson et al., 1991 and Marks et al., 1991, for example.

The antibodies, fragments, and derivatives of the presently disclosed subject matter can also include chimeric antibodies. As used herein in the context of antibodies, the term “chimeric”, and grammatical variants thereof, refers to antibody derivatives that have constant regions derived substantially or exclusively from antibody constant regions from one species and variable regions derived substantially or exclusively from the sequence of the variable region from another species. A particular kind of chimeric antibody is a “humanized” antibody, in which the antibodies are produced by substituting the complementarity determining regions (CDRs) of, for example, a mouse antibody, for the CDRs of a human antibody (see e.g., PCT International Patent Application Publication No. WO 1992/22653). Thus, in some embodiments, a humanized antibody has constant regions and variable regions other than the CDRs that are derived substantially or exclusively from the corresponding human antibody regions, and CDRs that are derived substantially or exclusively from a mammal other than a human.

The antibodies, fragments, and derivatives of the presently disclosed subject matter can also be single chain antibodies and single chain antibody fragments. Single-chain antibody fragments contain amino acid sequences having at least one of the variable regions and/or CDRs of the whole antibodies described herein but are lacking some or all of the constant domains of those antibodies. These constant domains are not necessary for antigen binding but constitute a major portion of the structure of whole antibodies.

Single-chain antibody fragments can overcome some of the problems associated with the use of antibodies containing a part or all of a constant domain. For example, single-chain antibody fragments tend to be free of undesired interactions between biological molecules and the heavy-chain constant region, or other unwanted biological activity. Additionally, single-chain antibody fragments are considerably smaller than whole antibodies and can therefore have greater capillary permeability than whole antibodies, allowing single-chain antibody fragments to localize and bind to target antigen-binding sites more efficiently. Also, antibody fragments can be produced on a relatively large scale in prokaryotic cells, thus facilitating their production. Furthermore, the relatively small size of single-chain antibody fragments makes them less likely to provoke an immune response in a recipient than whole antibodies. The single-chain antibody fragments of the presently disclosed subject matter include but are not limited to single chain fragment variable (scFv) antibodies and derivatives thereof such as, but not limited to tandem di-scFv, tandem tri-scFv, diabodies, and triabodies, tetrabodies, miniantibodies, and minibodies.

Fv fragments correspond to the variable fragments at the N-termini of immunoglobulin heavy and light chains. Fv fragments appear to have lower interaction energy of their two chains than Fab fragments. To stabilize the association of the V_{H} and V_{L} domains, they have been linked with peptides (see Bird et al., 1988; Huston et al., 1988), disulfide bridges (Glockshuber et al., 1990), and “knob in hole” mutations (Zhu et al., 1997). ScFv fragments can be produced by methods well known to those skilled in the art (see e.g., Whitlow et al., 1991 and Huston et al., 1993.

scFv can be produced in bacterial cells such as E. coli or in eukaryotic cells. One potential disadvantage of scFv is the monovalency of the product, which can preclude an increased avidity due to polyvalent binding, and their short half-life. Attempts to overcome these problems include bivalent (scFv′)₂ produced from scFv containing an additional C-terminal cysteine by chemical coupling (Adams et al., 1993; McCartney et al., 1995) or by spontaneous site-specific dimerization of scFv containing an unpaired C-terminal cysteine residue (see Kipriyanov et al., 1995).

Alternatively, scFv can be forced to form multimers by shortening the peptide linker to 3 to 12 residues to form multispecific antibodies, including but not limited to bispecific antibodies (sometimes referred to as “diabodies” (see Holliger et al., 1993). Reducing the linker still further can result in scFv trimers (trispecific antibodies, sometimes referred to as “triabodies”; see Kortt et al., 1997) and tetramers (tetraspecific antibodies, sometimes referred to as “tetrabodies”; see Le Gall et al., 1999). Construction of bivalent scFv molecules can also be achieved by genetic fusion with protein dimerizing motifs to form “miniantibodies” (see Pack et al., 1992) and “minibodies” (see Hu et al., 1996). scFv-scFv tandems ((scFv)₂) can be produced by linking two scFv units by a third peptide linker (see Kurucz et al., 1995).

A particular type of multimeric scFv is a “bispecific T-cell engager” (BiTe; see e.g., Halland et al., 2020) which is a binding molecule in which one of the specificities engages a T cell and/or an immune effector cell and another specific binds to an antigen of interest. BiTes are described, for example, in U.S. Pat. Nos. 9,988,452; 10,113,003; 10,183,992; and 10,358,492, each of which is incorporated by reference herein in its entirety. In some embodiments, a BiTe includes a specificity that binds to a CD3 molecule.

Bispecific diabodies can be produced through the non-covalent association of two single chain fusion products consisting of V_{H} domain from one antibody connected by a short linker to the V_{L} domain of another antibody (see Kipriyanov et al., 1998). The stability of such bispecific diabodies can be enhanced by the introduction of disulfide bridges or “knob in hole” mutations as described hereinabove or by the formation of single chain diabodies (scDb) wherein two hybrid scFv fragments are connected through a peptide linker (see Kontermann et al., 1999).

Tetravalent bispecific molecules can be produced, for example, by fusing an scFv fragment to the CH₃ domain of an IgG molecule or to a Fab fragment through the hinge region (see Coloma et al., 1997). Alternatively, tetravalent bispecific molecules have been created by the fusion of bispecific single chain diabodies (see Alt et al., 1999). Smaller tetravalent bispecific molecules can also be formed by the dimerization of either scFv-scFv tandems with a linker containing a helix-loop-helix motif (DiBi miniantibodies; see Muller et al., 1998) or a single chain molecule comprising four antibody variable domains (V_{H} and V_{L}) in an orientation preventing intramolecular pairing (tandem diabody; see Kipriyanov et al., 1999).

Bispecific F(ab′)₂ fragments can be created by chemical coupling of Fab′ fragments or by heterodimerization through leucine zippers (see Shalaby et al., 1992; Kostelny et al., 1992). Also available are isolated V_{H} and V_{L} domains (see U.S. Pat. Nos. 6,172,197; 6,248,516; and 6,291,158).

The presently disclosed subject matter also includes functional equivalents of anti-gastrin antibodies. As used herein, the phrase “functional equivalent” as it refers to an antibody refers to a molecule that has binding characteristics that are comparable to those of a given antibody. In some embodiments, chimerized, humanized, and single chain antibodies, as well as fragments thereof, are considered functional equivalents of the corresponding antibodies upon which they are based.

Functional equivalents also include polypeptides with amino acid sequences substantially the same as the amino acid sequence of the variable or hypervariable regions of the antibodies of the presently disclosed subject matter. As used herein with respect to amino acid sequences, the phrase “substantially the same” refers to a sequence with, in some embodiments at least 80%, in some embodiments at least 85%, in some embodiments at least about 90%, in some embodiments at least 91%, in some embodiments at least 92%, in some embodiments at least 93%, in some embodiments at least 94%, in some embodiments at least 95%, in some embodiments at least 96%, in some embodiments at least 97%, in some embodiments at least 98%, and in some embodiments at least about 99% sequence identity to another amino acid sequence, as determined by the FASTA search method in accordance with Pearson & Lipman, 1988. In some embodiments, the percent identity calculation is performed over the full length of the amino acid sequence of an antibody of the presently disclosed subject matter.

Functional equivalents further include fragments of antibodies that have the same or comparable binding characteristics to those of a whole antibody of the presently disclosed subject matter. Such fragments can contain one or both Fab fragments, the F(ab′)2 fragment, the F(ab′) fragment, an Fv fragment, or any other fragment that includes at least one antigen binding domain. In some embodiments, the antibody fragments contain all six CDRs of a whole antibody of the presently disclosed subject matter, although fragments containing fewer than all of such regions, such as three, four, or five CDRs, can also be functional equivalents as defined herein. Further, functional equivalents can be or can combine members of any one of the following immunoglobulin classes: IgG, IgM, IgA, IgD, and IgE, and the subclasses thereof, as well as other subclasses as might be appropriate for non-mammalian subjects (e.g., IgY for chickens and other avian species).

Functional equivalents further include peptides that have the same or comparable characteristics to those of a whole protein of the presently disclosed subject matter. Such peptides can contain one or more antigens of the whole protein, which can elicit an immune response in the treated subject.

Functional equivalents also include aptamers and other non-antibody molecules, provided that such molecules have the same or comparable binding characteristics to those of a whole antibody of the presently disclosed subject matter.

The term “comprising”, which is synonymous with “including” “containing”, or “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting of” excludes any element, step, or ingredient not specifically recited. It is noted that, when the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of the related disclosure or claim to the specified materials and/or steps, plus those that do not materially affect the basic and novel characteristic(s) of the disclosed and/or claimed subject matter. For example, a pharmaceutical composition can “consist essentially of” a pharmaceutically active agent or a plurality of pharmaceutically active agents, which means that the recited pharmaceutically active agent(s) is/are the only pharmaceutically active agent(s) present in the pharmaceutical composition. It is noted, however, that carriers, excipients, and/or other inactive agents can and likely would be present in such a pharmaceutical composition and are encompassed within the nature of the phrase “consisting essentially of”.

With respect to the terms “comprising”, “consisting of”, and “consisting essentially of”, where one of these three terms is used herein, the presently disclosed and claimed subject matter can include the use of either of the other two terms. For example, in some embodiments, the presently disclosed subject matter relates to compositions comprising antibodies. It would be understood by one of ordinary skill in the art after review of the instant disclosure that the presently disclosed subject matter thus encompasses compositions that consist essentially of the antibodies of the presently disclosed subject matter, as well as compositions that consist of the antibodies of the presently disclosed subject matter.

As used herein, the phrase “immune cell” refers to the cells of a mammalian immune system including but not limited to antigen presenting cells, B cells, basophils, cytotoxic T cells, dendritic cells, eosinophils, granulocytes, helper T cells, leukocytes, lymphocytes, macrophages, mast cells, memory cells, monocytes, natural killer cells, neutrophils, phagocytes, plasma cells, γδ T cells, NKT cells, and mucosal-associated invariant T (MATT) cells.

As used herein, the phrase “immune response” refers to immunities including but not limited to innate immunity, humoral immunity, cellular immunity, immunity, inflammatory response, acquired (adaptive) immunity, autoimmunity, and/or overactive immunity.

As used herein, the phrase “gastrin-associated cancer” is in some embodiments a tumor or cancer or a cell therefrom in which a gastrin gene product acts as a trophic hormone to stimulate tumor and/or cancer cell growth both when exogenously applied to tumor and/or cancer cells and also in vivo through autocrine and paracrine mechanisms. Exemplary gastrin-associated cancers include pancreatic cancer, gastric cancer, gastroesophageal cancer, and colorectal cancer. In some embodiments, a gastrin-associated cancer” is a tumor or cancer or a cell therefrom that itself produces gastrin, which in some embodiments can result in a hormonal and/or feedback effect on the growth of the tumor, cancer, and/or a cell therefrom (e.g., a gastrinoma) and/or is a gastrin-responsive tumor and/or cancer (e.g., a tumor and/or cancer, or a cell therefrom, the growth of which is stimulated by gastrin and/or gastrin signaling through a gastrin recetor (e.g., the CCK-B receptor).

The term “polynucleotide” as used herein includes but is not limited to DNA, RNA, complementary DNA (cDNA), messenger RNA (mRNA), ribosomal RNA (rRNA), small hairpin RNA (shRNA), small nuclear RNA (snRNA), short nucleolar RNA (snoRNA), microRNA (miRNA), genomic DNA, synthetic DNA, synthetic RNA, and/or tRNA.

As used herein, the phrases “single chain variable fragment”, “single-chain antibody variable fragments”, and “scFv” antibodies refer to forms of antibodies comprising the variable regions of only the heavy and light chains, connected by a linker peptide.

The term “subject” as used herein refers to a member of any invertebrate or vertebrate species. Accordingly, the term “subject” is intended to encompass in some embodiments any member of the Kingdom Animalia including, but not limited to the phylum Chordata (e.g., members of Classes Osteichythyes (bony fish), Amphibia (amphibians), Reptilia (reptiles), Ayes (birds), and Mammalia (mammals), and all Orders and Families encompassed therein.

Thus, the compositions and methods of the presently disclosed subject matter are particularly useful for warm-blooded vertebrates. Thus, in some embodiments the presently disclosed subject matter concerns mammals and birds. More particularly provided are compositions and methods derived from and/or for use in mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also provided is the use of the disclosed methods and compositions on livestock, including but not limited to domesticated swine (pigs and hogs), ruminants, horses, poultry, and the like.

As used herein, the terms “T cell” and “T lymphocyte” are interchangeable and used synonymously. Examples include, but are not limited to, naive T cells, central memory T cells, effector memory T cells, cytotoxic T cells, T regulatory cells, helper T cells and combinations thereof.

As used herein, the phrase “therapeutic agent” refers to an agent that is used to, for example, treat, inhibit, prevent, mitigate the effects of, reduce the severity of, reduce the likelihood of developing, slow the progression of, and/or cure, a disease or disorder such as but not limited to a gastrin-associated tumor and/or cancer.

The terms “treatment” and “treating” as used herein refer to both therapeutic treatment and prophylactic or preventative measures, wherein the object is to prevent or slow down (lessen) the targeted pathologic condition, prevent the pathologic condition, pursue or obtain beneficial results, and/or lower the chances of the individual developing a condition, disease, or disorder, even if the treatment is ultimately unsuccessful. Those in need of treatment include those already with the condition as well as those prone to have or predisposed to having a condition, disease, or disorder, or those in whom the condition is to be prevented.

As used herein, the term “tumor” refers to any neoplastic cell growth and/or proliferation, whether malignant or benign, and all pre-cancerous and cancerous cells and tissues the initiation, progression, growth, maintenance, of metastasis of which is directly or indirectly influenced by autocrine and/or paracrine action of gastrin. The terms “cancer” and “tumor” are used interchangeably herein and can refer to both primary and metastasized solid tumors and carcinomas of any tissue in a subject, including but not limited to pancreatic cancer, gastric cancer, gastroesophageal cancer, and colorectal cancer (referred to herein collectively as “gastrin-associated” tumors and/or cancers). As used herein, the terms “cancer and “tumor” are also intended to refer to multicellular tumors as well as individual neoplastic or pre-neoplastic cells. In some embodiments, a cancer or a tumor comprises a cancer or tumor of an epithelial tissue such as, but not limited to a carcinoma. In some embodiments, a tumor is an adenocarcinoma, which in some embodiments is an adenocarcinoma of the pancreas, liver, stomach, esophagus, colon, or rectum, and/or a metastatic cell derived therefrom. In some embodiments, a tumor and/or a cancer is associated with fibrosis, meaning that as a direct or indirect consequence of the development of the tumor and/or the cancer, one or more regions of fibrosis typically develop in the area of the tumor and/or the cancer.

All genes, gene names, and gene products disclosed herein are intended to correspond to homologs and/or orthologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the gastrin gene products presented in GENBANK® biosequence database Accession Nos: NM_000805.5 and NP_000796.1 (SEQ ID NOs: 11 and 12, respectively), the human nucleic acid and amino acid sequences disclosed are intended to encompass homologous and orthologous gastrin genes and gene products from other animals including, but not limited to other mammals, fish, amphibians, reptiles, and birds. Also encompassed are any and all nucleotide sequences that encode gastrin amino acid sequences, including but not limited to those disclosed in the corresponding GENBANK® entries (e.g., NP_000796.1 and NM_000805.5; SEQ ID NOs: 11 and 12, respectively).

### II. Compositions Comprising Polyclonal Antibody Stimulator (PAS)

The gastrin vaccine Polyclonal Antibody Stimulator (PAS) comprises in some embodiments a 9-amino acid gastrin epitope derived from the N-terminal sequence of gastrin that is identical in mice and humans and is conjugated to diphtheria toxoid (DT) through a linker molecule. This conjugate has been formulated in an oil-based adjuvant to create PAS. PAS stimulates the production of specific and high-affinity polyclonal anti-gastrin antibodies, whereas DT alone had no effect (Watson et al., 1996). Preclinical studies were performed in several animal models with gastrointestinal (GI) cancer that are gastrin responsive, including colon cancer (Singh et al., 1986; Smith & Solomon, 1988; Upp et al., 1989; Smith et al., 1996), gastric cancer (Smith et al., 1998; Watson et al., 1989), lung cancer (Rehfeld et al., 1989), and pancreatic cancer (Smith et al., 1990; Smith et al., 1991; Smith et al., 1995; Segal et al., 2014).

In animals, PAS-generated anti-gastrin antibodies have been shown to reduce the growth and metastasis of gastrointestinal tumors (Watson et al., 1995; Watson et al., 1996; Watson et al., 1999a). Both active immunizations with PAS and passive immunization with PAS-generated anti-gastrin antibodies (Watson et al., 1999a) have been shown to inhibit tumor growth in animal models of GI cancers (Watson et al., 1998; Watson et al., 1999a).

PAS has also shown significant promise in improving survival in gastric cancer in Phase 2 clinical trials and in pancreatic cancer in Phase 2 and Phase 3 clinical trials. PAS vaccination has been shown to elicit a humoral immune response as demonstrated by the production of neutralizing antibodies to gastrin. By eliminating gastrin, the vaccine slows tumor growth and has potential to provide long-term tumor killing activity. PAS administration generates a humoral antibody response and a cellular immune response to the onco-fetal protein gastrin, which is inappropriately expressed (i.e., overexpressed) in various tumors and/or cancers including but not limited to various adenocarcinomas such as gastric adenocarcinoma and PDAC. This inappropriate gastrin expression causes an autocrine and paracrine growth-promoting effect. PAS administration with its subsequent generation of humoral antibodies to gastrin, will help eliminate this pathological growth-promoting effect. In addition, a PAS-mediated humoral immune response to gastrin will also help reverse the promotion of angiogenesis, circumvention of apoptosis, increase in cell migration, and increase in invasive enzyme expression that are associated with inappropriate gastrin expression (Watson et al., 2006).

In some embodiments, PAS comprises 3 subunits. The first subunit is a gastrin epitope, which in some embodiments is a peptide that comprises amino-terminal amino acid residues 1-9 of human G17 with a carboxy-terminal seven (7) amino acid spacer sequence that terminates in a cysteine residue. An exemplary sequence for this first subunit is EGPWLEEEE (SEQ ID NO: 2).

The second subunit of PAS is a linker that covalently links the first subunit to the third subunit. In some embodiments, the linker is a ε-maleimido caproic acid N-hydroxysuccinamide ester (eMCS), although any linker, including non-peptide linkers such as but not limited to polyethylene glycol linkers, could be used for this purpose.

The third subunit of PAS is a diphtheria toxoid (DT), which is used as a carrier protein to enhance a humoral response directed against the first subunit (in particular, a humoral response directed against the gastric epitope). It is noted, however, that in some embodiments carrier proteins other than diphtheria toxoid could be employed such as but not limited to tetanus toxoid or bovine serum albumin.

In some embodiments, the three subunits are formulated for intramuscular (i.m.) injection, and the formulation has excellent physical, chemical, and pharmaceutical properties. PAS also elicits a B cell response with generation of neutralizing antibodies to gastrin. This is relevant in cancer, since gastrin increases cellular proliferation, promotes angiogenesis, facilitates circumvention of apoptosis, increases cell migration, increases invasive enzyme expression, and is associated with fibrosis on certain tumor microenvironments (e.g., in PDAC). In accordance with some aspects of the presently disclosed subject matter, if the actions of gastrin are blocked, CD8⁺ lymphocytes influx into tumors, rendering them more likely to respond to immunotherapy (e.g., a T-cell mediated response). As disclosed herein. PAS also elicits a T cell response and CD8⁺ cells that produce cytokines in response to gastrin stimulation.

PAS can be designed as a therapeutic vaccine or immunotherapeutic. PAS-induced humoral antibodies are highly specific and typically characterized by high affinity to G17 and Gly-G17.

PAS consistently induced therapeutically efficacious levels of antibodies that are directed against gastrin. Twenty-two clinical studies have been completed with a total of 1,542 patients. Importantly, treatment with PAS demonstrated an excellent safety and tolerability profile, and further resulted in a survival benefit in colorectal, gastric, and pancreatic cancer patients. Used as a monotherapy, an exemplary dose and schedule were identified to be 250 μg/0.2 ml dosed at 0, 1, and 3 weeks.

Taken collectively, the conclusions that can be made from the 22 studies and >1,500 patients treated with PAS are as follows:
(a) Nonclinical data demonstrated both in vitro and in vivo anti-tumor efficacy of anti-G17 antibodies, with a wide therapeutic index in various cancer models, including human pancreatic cancer models;
(b) PAS can be administered at very safe and well tolerated doses, and effectively causes a B cell antibody response to gastrin with no adverse reactions and no induction of negative autoimmune effects; and
(c) Numerous clinical studies have demonstrated a survival benefit across gastrointestinal tumors, including pancreatic cancer, and a correlation between generation of anti-G17 antibody response and improved survival.

However, clinical studies have also demonstrated that there were long term survivors, which suggested that additional therapeutic benefits also resulted from PAS administration. While not wishing to be bound by any particular theory of operation, it is possible that PAS treatment might have also induced a T cell immune response characterized by activation of cytotoxic T cells and memory cells in these subjects.

### III. CARs and Methods of Producing the Same

Chimeric antigen receptors (CARs) are artificially constructed hybrid proteins or polypeptides containing the antigen binding domains of an antibody (such as, but not limited to an scFv) linked to one or more T-cell signaling domains. Characteristics of CARs include their ability to redirect T-cell specificity and reactivity toward a selected target in a non-MHC-restricted manner, thereby exploiting the antigen-binding properties of monoclonal antibodies. The non-MHC-restricted antigen recognition gives T cells expressing CARs the ability to recognize antigen independent of antigen processing, thus bypassing a major mechanism of tumor escape. Moreover, when expressed in T-cells, CARs advantageously do not dimerize with endogenous T cell receptor (TCR) alpha and beta chains. CARs and methods to prepare the same are described generally in U.S. Pat. Nos. 6,410,319; 8,389,282; and 10,059,923; as well as U.S. Patent Application Publication Nos. 2007/0036773, 2009/0180989, 2009/0257991, 2011/0038836, 2012/0058051, 2012/0213783, and 2012/0252742, each of which is incorporated herein by reference in its entirety.

As used herein, the phrases “have antigen specificity” and “elicit antigen-specific response” mean that a CAR can specifically bind to and immunologically recognize an antigen, in some embodiments a tumor-associated antigen (TAA), a cancer-associated antigen (CAA), or an immunogenic epitope thereof, such that binding of the CAR to the antigen elicits an immune response.

As used herein, the phrase “antigen-specific targeting region” (ASTR) refers to the region of a CAR that targets (i.e., binds to) specific antigens and/or epitopes. The CARs of the presently disclosed subject matter comprise in some embodiments one ASTR (i.e., are monospecific) and in some embodiments comprise two targeting regions which target two different antigens and/or epitopes (i.e., are bispecific). In some embodiments, CARs comprise three or more targeting regions which target at least three or more different antigens (i.e., are trispecific or multispecific). The targeting regions on the CAR are extracellular. In some embodiments, the antigen-specific targeting regions comprise an antibody or a functional equivalent thereof or a fragment thereof or a derivative thereof, and in some embodiments each of the targeting regions targets a different antigen or epitope. The targeting regions can comprise full length heavy chain, Fab fragments, single chain FIT (scFv) fragments, divalent single chain antibodies or diabodies, each of which are specific to the target antigen. There are, however, numerous alternatives, such as linked cytokines (which leads to recognition of cells bearing the cytokine receptor), affibodies, ligand binding domains from naturally occurring receptors, soluble protein/peptide ligand for a receptor (for example on a tumor cell), peptides, and vaccines to prompt an immune response, which may each be used in various embodiments of the presently disclosed subject matter. In fact, almost any molecule that binds a given antigen with high affinity can be used as an ASTR, as will be appreciated by those of skill in the art.

Thus, as used herein, the terms “Chimeric Antigen Receptor”, “CAR”, or “CARs” refer to engineered receptors, which graft an antigen specificity onto cells (for example T cells such as naive T cells, central memory T cells, effector memory T cells or combination thereof). CARs are also known as artificial T-cell receptors, chimeric T-cell receptors, or chimeric immunoreceptors. The CARs of the presently disclosed subject matter comprise one or more ASTRs, an extracellular domain, a transmembrane domain, one or more co-stimulatory domains, and an intracellular signaling domain. In those embodiments where two or more ASTRs are present, the two or more ASTRs can target at least two different antigens and can be arranged in tandem and separated by linker sequences. In some embodiments, the extracellular spacer domain is optional. In some embodiments, the CAR is a monospecific CAR that targets an antigen or epitope associated with a tumor, which in some embodiments can be a tumor characterized by fibrosis.

In some embodiments, a CAR comprises an ASTR that binds to a gastrin gene product or a receptor for which gastrin is a ligand. Exemplary antibodies that bind to gastrin are described in U.S. Pat. Nos. 7,235,376 and 8,808,695 (each of which is incorporated herein by reference in its entirety), including but not limited to the monoclonal antibodies produced by hybridoma 490-1 (ATCC Accession No. PTA-6189), hybridoma 491-1 (ATCC PTA-6190), and hybridoma 495-1 (ATCC Accession No. PTA-6191). Exemplary antibodies that bind to gastrin receptor CCK-B are described in U.S. Pat. No. 8,388,966 and U.S. Patent Application Publication No. 2003/0086941 (each of which is incorporated herein by reference in its entirety). In some embodiments, a paratope and/or paratope-containing antibody fragment from any of these antibodies can be incorporated into a CAR, which in some embodiments can then be incorporated into a CAR-T cell, for use in the compositions and methods of the presently disclosed subject matter. In some embodiments, a CAR incorporates a paratope and/or paratope-containing antibody fragment from a commercially available anti-CCK-B antibody, such as but not limited to those sold by ThermoFisher Scientific (e.g., Catalog Nos. PAS-84814, PAS-103116, PAS-32348, and others), Creative Biolabs (e.g., Catalog Nos. MOB-2496CT, MOB-3741z-S(P), MOB-3741z-F(E), and MOB-3741z), LSBio (e.g., Catalog No. LS-C128133-20), Novus Biologicals (e.g., Catalog No. NLS6535), Sigma-Aldrich (e.g., Catalog No. SAB1402711), Abcam (e.g., Catalog Nos. ab27441 and ab83180), and others.

As used herein, the phrase “co-stimulatory domain” (CSD) refers to the portion of the CAR that enhances the proliferation, survival, and/or development of memory cells. The CARs of the presently disclosed subject matter can comprise one or more co-stimulatory domains. In some embodiments, each co-stimulatory domain comprises the costimulatory domain of one or more of members of the TNFR superfamily, CD28, CD137 (4-1BB), CD134 (OX40), Dap10, CD27, CD2, CDS, ICAM-1, LFA-1 (CD11a/CD18), Lck, TNFR-I, TNFR-II, Fas, CD30, CD40, or any combinations thereof. Other co-stimulatory domains (e.g., from other proteins) will be apparent to those of skill in the art and can be used in connection with alternate embodiments of the presently disclosed subject matter.

As used herein, the phrase “extracellular spacer domain” (ESD) refers to the hydrophilic region that is between the ASTR and the transmembrane domain. In some embodiments, the CARs of the presently disclosed subject matter comprise an extracellular spacer domain. In some embodiments, the CARs of the presently disclosed subject matter do not comprise an extracellular spacer domain. The extracellular spacer domains can include, but are not limited to, Fc fragments of antibodies or fragments or derivatives thereof, hinge regions of antibodies or fragments or derivatives thereof, CH2 regions of antibodies, CH3 regions of antibodies, artificial spacer sequences, or combinations thereof. Examples of extracellular spacer domains include, but are not limited to, CD8a hinge, and artificial spacers made of polypeptides which can be as small as, for example, Gly3 or CH1 and CH3 domains of IgGs (such as but not limited to human IgG4). In some embodiments, the extracellular spacer domain is any one or more of (i) a hinge, CH2, and CH3 regions of IgG4; (ii) a hinge region of IgG4; (iii) a hinge and CH2 of IgG4; (iv) a hinge region of CD8α; (v) a hinge, CH2, and CH3 regions of IgG1; (vi) a hinge region of IgG1; (vi) a hinge and CH2 region of IgG1; and/or (vii) a hinge region of IgD. Other extracellular spacer domains will be apparent to those of skill in the art and may be used in connection with any embodiments of the presently disclosed subject matter.

In some embodiments, the binding domain of a CAR of the presently disclosed subject matter is followed by a hinge region, which refers to the region that moves the antigen binding domain away from the effector cell surface to enable proper cell/cell contact, antigen binding, and activation (see e.g., Patel et al., 1999). In some embodiments, a hinge region is an immunoglobulin hinge region and can be a wild type immunoglobulin hinge region or a modified immunoglobulin hinge region. Other exemplary hinge regions used in the CARs described herein include the hinge region derived from the extracellular regions of type 1 membrane proteins such as CD8α, CD4, CD28, and CD7, which can be wild type hinge regions from these molecules or can be modified. In some embodiments, a hinge region is an IgD hinge region or a subsequence thereof.

As used herein, the phrase “modified hinge region” refers to (a) a wild type hinge region with in some embodiments up to 30% amino acid changes (e.g., up to 25% amino acid changes, up to 20% amino acid changes, up to 15% amino acid changes, up to 10% amino acid changes, or up to 5% amino acid changes, including but not limited to amino acid substitutions, additions, and/or deletions); (b) a portion of a wild type hinge region that is in some embodiments at least 10 amino acids in length (e.g., at least 11, 12, 13, 14, 15, 16, 17, 18, 19, 20, 25, 30, 35, 40, 45, 50, or more amino acids) in length with in some embodiments up to 30% amino acid changes (e.g., up to 25% amino acid changes, up to 20% amino acid changes, up to 15% amino acid changes, up to 10% amino acid changes, or up to 5% amino acid changes, including but not limited to amino acid substitutions, additions, and/or deletions); or (c) a portion of a wild type hinge region that comprises the core hinge region (which in some embodiments can be 4, 5, 6, 7, 8, 9, 10, 11, 12, 13, 14, or or at least 4, 5, 6, 7, 8, 9, 10, 11, 12, 13, 14, or 15 amino acids in length). When a modified hinge region is interposed between and connecting a binding domain and another region (e.g., a transmembrane domain) in the CARs described herein, it allows the chimeric fusion protein to maintain specific binding to its target (e.g., to a tumor-associated antigen or epitope).

As used herein, the phrase “intracellular signaling domain” (ISD) or “cytoplasmic domain” refer to the portion of the CAR which transduces the effector function signal and directs the cell to perform its specialized function. Examples of domains that transduce the effector function signal include but are not limited to the zeta chain of the T-cell receptor complex or any of its homologs (e.g., the eta chain, FcεR1 γ and β chains, MB1 (Igα) chain, B29 (Igβ) chain, etc.), human CD3 zeta chain, CD3 polypeptides (delta and epsilon), syk family tyrosine kinases (Syk, ZAP 70, etc.), src family tyrosine kinases (Lck, Fyn, Lyn, etc.), and other molecules involved in T-cell transduction, such as CD2, CD5, and CD28. Other intracellular signaling domains will be apparent to those of skill in the art and can be used in connection with any embodiments of the presently disclosed subject matter.

As used herein, the phrases “linker”, “linker domain”, and “linker region” refer to an oligo- or polypeptide region from about 1 to 100 amino acids in length, which links together any of the domains and/or regions of a CAR of the presently disclosed subject matter. In some embodiments, linkers comprise, consist essentially of, or consist of flexible residues like glycine and serine so that the adjacent protein domains are free to move relative to one another. Longer linkers can be used when it is desirable to ensure that two adjacent domains do not sterically interfere with one another, such as can be the case with bispecific, trispecific, and multispecific CARs. Linkers can be cleavable or non-cleavable. Examples of cleavable linkers include 2A linkers (for example T2A; see U.S. Pat. No. 8,802,374, incorporated herein by reference in its entirety), 2A-like linkers, or functional equivalents thereof, and combinations thereof. In some embodiments, the linkers include the picornaviral 2A-like linker, cis-acting hydrolase element (CHYSEL) sequences of porcine teschovirus (P2A), Thosea asigna virus (T2A), or combinations, variants, and functional equivalents thereof. In some embodiments, the linker sequences can comprise Asp-Val/Ile-Glu-X-Asn-Pro-Gly2A-Pro2B motif, which results in cleavage between the 2A glycine and the 2B proline. Other linkers will be apparent to those of skill in the art and may be used in connection with any embodiments of the presently disclosed subject matter.

As used herein, the phrase “transmembrane domain” (TMD or TD) refers to the region of the CAR that crosses the plasma membrane. The transmembrane domains of the CARs of the presently disclosed subject matter are the transmembrane regions of a transmembrane protein (for example Type I transmembrane proteins), an artificial hydrophobic sequence, or a combination thereof. Other transmembrane domains will be apparent to those of skill in the art and can be used in connection with any embodiments of the presently disclosed subject matter.

CARs and the T cells that have been modified to express CARs can be described as being “first generation”, “second generation”, “third generation”, or “fourth generation” based on the various components that are present in the CARs. “First generation” CARs include an antigen binding domain, transmembrane domain, and an intracellular domain, typically a CD3zeta intracellular domain. “Second generation” CARs further comprise a costimulatory domain. “Third generation” CARs further comprise other signaling domains, such as but not limited to 4-IBB signaling domains and/or OX40 signaling domains. “Fourth generation” CAR T cells typically are characterized by the presence of a second or third generation CAR, and have been further modified to express proliferative cytokines (e.g., IL-12; Pegram et al., 2012) or additional costimulatory ligands (e.g., 4-1BBL; Stephan et al., 2007).

The presently disclosed subject matter thus provides in some embodiments CARs that bind to antigens present within tumors such as, but not limited to tumor-associated antigens and/or epitopes, meaning that the antigens and/or epitopes to which the presently disclosed CARs bind are expressed by tumor cells but are not expressed by non-tumor cells.

In some embodiments, the presently disclosed subject matter provides nucleic acid molecules encoding chimeric antigen receptors (CARs) that are directed against tumor-associated epitopes. The nucleic acids can in some embodiments be DNA and/or RNA, optionally mRNA encoding the CARs. In some embodiments, the nucleic acid molecules encode a CAR comprising an antibody or antibody fragment that includes a binding domain, a transmembrane domain, and an intracellular signaling domain comprising a stimulatory domain, wherein the binding domain binds to a tumor-associated antigen (TAA) or epitope.

CARs also comprise a transmembrane domain (TD), and in some embodiments the TD of a presently disclosed CAR is a TD of a protein selected from the group consisting of a T cell receptor (TCR) alpha chain, a TCR beta chain, a TCR zeta chain, CD28, CD3 epsilon, CD45, CD4, CD5, CD8, CD9, CD16, CD22, CD33, CD37, CD64, CD80, CD86, CD134, CD137, and CD154. In some embodiments, the TD comprises amino acids sequence FWVLVVVGGVLACYSLLVTVAFIIFWV (SEQ ID NO: 5) or the amino acid sequence FWALVVVAGVLFCYGLLVTVALCVIWT (SEQ ID NO: 6), which correspond to the transmembrane domains of the human and mouse CD28 molecules, respectively. As a TD generally serves only to anchor the CAR in the membrane of a cell expressing the CAR, modifications in the sequences of known TDs are also permitted, provided that the modifications do not destroy the ability of the TD to function as a TD. Thus, in some embodiments, the TD comprises an amino acid sequence having at least one, two, or three but not more than 20, 10, or 5 modifications of the amino acid sequence FWVLVVVGGVLACYSLLVTVAFIIFWV (i.e., amino acids 34-60 of the human T-cell-specific surface glycoprotein CD28 precursor of GENBANK® Accession No. NP_001230007.1; SEQ ID NO: 5) or the amino acid sequence FWALVVVAGVLFCYGLLVTVALCVIWT (i.e., amino acids 151-177 of the mouse T-cell-specific surface glycoprotein CD28 precursor of GENBANK® Accession No. NP_031668.3; SEQ ID NO: 6), or comprises an amino acid sequence with at least 95% identity thereto.

The nucleic acid molecules of the presently disclosed subject matter can in some embodiments encode an anti-TAA binding domain that is connected to the TD by an extracellular hinge region and/or the TD connected to the intracellular domains via an intracellular hinge regions. Extracellular hinge regions provide the CARs with flexibility between the binding domain and the TD, and in some embodiments can influence cytokine secretion and cell-mediated killing of target cells by the CARs (Sadelain et al., 2009). Non-limiting examples of extracellular and intracellular hinge regions that can be employed in a CAR include Fc regions of immunoglobulins and immunoglobulin-like domains from CD8a or CD28 (e.g., the human CD8 and CD28 extracellular and intracellular hinges disclosed in U.S. Pat. No. 8,465,743). In some embodiments, the presently disclosed CARs comprise a hinge region, which in some embodiments comprises a hinge region of an immunoglobulin delta chain including, but not limited to amino acids of 101-158 of GENBANK® Accession No. AAA52771.1 (SEQ ID NO: 13), or a subsequence thereof.

CARs also comprise intracellular domains that generally include one or more costimulatory domains and one or more signaling domains. As such, the isolate nucleic acid molecules of the presently disclosed subject matter further comprise a sequence encoding a costimulatory domain. In some embodiments, the costimulatory domain comprises the intracellular domain of a costimulatory molecule selected from the group consisting of CD27, CD28, 4-1BB, OX40, CD30, CD40, PD-1, ICOS, lymphocyte function-associated antigen-1 (LFA-1), CD7, LIGHT, NKG2C, B7-H3, a ligand that specifically binds with CD83, and any combination thereof. A particular non-limiting example of a costimulatory domain of the presently disclosed subject matter comprises an amino acid sequence derived from the human or mouse CD28 polypeptide, including but not limited to amino acids 123-220 of GENBANK® Accession No NP_006130.1 (SEQ ID NO: 14) or amino acids 124-218 of GENBANK® Accession No NP_031668.3 (SEQ ID NO: 15). Modifications of the costimulatory domains that do not significantly impact the ability of the domain to function as a costimulatory domain are also permitted. Thus, in some embodiments the encoded costimulatory domain comprises an amino acid sequence having at least one, two, or three but not more than 20, 10, or 5 modifications of an amino acid sequence of any of amino acids 123-220 of GENBANK® Accession No NP_006130.1 (SEQ ID NO: 14) or amino acids 124-218 of GENBANK® Accession No NP_031668.3 (SEQ ID NO: 15), or comprises an amino acid sequence with at least 95% identity thereto.

The nucleic acid molecules of the presently disclosed subject matter also encode CARs that comprise at least one intracellular signaling domain. Exemplary, non-limiting intracellular signaling domains include those derived from 4-1BB and/or from CD3zeta. More particularly, an intracellular signaling domain can comprise a human CD3zeta intracellular signaling domain comprising amino acids of 52-163 of GENBANK® Accession No. NP_000725.1 (SEQ ID NO: 16) or a mouse CD3zeta intracellular signaling domain comprising amino acids 52-164 of GENBANK® Accession No. NP_001106862.1 (SEQ ID NO: 17). Here as well, modifications in the sequences of intracellular signaling domains are also permitted, provided that the modifications do not destroy the ability of the intracellular signaling domains to function as an intracellular signaling domain. As such, in some embodiments the encoded intracellular signaling domain comprises an amino acid sequence having at least one, two, or three but not more than 20, 10 or 5 modifications of an amino acid sequence as set forth in amino acids of 52-163 of GENBANK® Accession No. NP_000725.1 (SEQ ID NO: 16) or amino acids 52-164 of GENBANK® Accession No. NP_001106862.1 (SEQ ID NO: 17), or comprises a amino acid sequence with at least 95% identity thereto.

In some embodiments, a costimulatory domain is fused to an intracellular signaling domain to create an intracellular domain with dual functions. By way of example and not limitation, one of the costimulatory domains disclosed herein can be fused in frame with one of the intracellular signaling domains disclosed herein. In some embodiments, the intracellular domain comprises amino acids 123-220 of GENBANK® Accession No. NP_006130.1 (SEQ ID NO: 14) fused to amino acids 52-163 of GENBANK® Accession No. NP_000725.1 (SEQ ID NO: 16), wherein the sequences comprising the intracellular domain are expressed in the same frame and as a single polypeptide chain.

CARs can also comprise a leader sequence, such as but not limited to a CD8 leader sequence. Non-limiting examples of CD8 leader sequences that can be included in a CAR are the human CD8 leader sequence corresponding to amino acids 1-21 of Accession No. NP_741969.1 (SEQ ID NO: 18) of the GENBANK® biosequence database.

In some embodiments, an nucleic acid sequence encoding a chimeric antigen receptor (CAR) of the presently disclosed subject matter comprises a (i) binding domain that binds to a tumor-exclusive epitope of a human MUC1 polypeptide and (ii) a CD3 zeta signaling domain. In some embodiments, the nucleic acid further encodes a costimulatory signaling domain, which in some embodiments is selected from the group consisting of a CD28 Signaling Domain and a 4-1BB Signaling Domain.

As such, the presently disclosed subject matter provides chimeric antigen receptor (CAR) molecules that in some embodiments comprise a binding domain, a transmembrane domain (TD), and an intracellular domain, wherein the binding domain binds to a TAA or an epitope thereof. In some embodiments, the binding domain is a subsequence of an antibody that binds to a TAA or an epitope thereof of a claudin18.2 antigen, a glypican3 antigen, a mesothelin antigen, a carcinoembryonic antigen (CEA), a prostate stem cell antigen (PSCA), and a CD70 antigen, or a fragment thereof comprising a paratope of the antibody, optionally wherein the binding domain is human or humanized. In some embodiments, the binding domain is a scFv.

In some embodiments of the presently disclosed CAR molecules, the TD comprises a TD of a protein selected from the group consisting of the T-cell receptor (TCR) alpha chain, the TCR beta chain, the TCR zeta chain, CD28, CD3 epsilon, CD45, CD4, CD5, CD8, CD9, CD16, CD22, CD33, CD37, CD64, CD80, CD86, CD134, CD137, and CD154. In some embodiments, the TD comprises: (i) amino acids 34-60 of the human T-cell-specific surface glycoprotein CD28 precursor of GENBANK® Accession No. NP_001230007.1 or amino acids 151-177 of the mouse T-cell-specific surface glycoprotein CD28 precursor of GENBANK® Accession No. NP_031668.3 (SEQ ID NO: 15); (ii) amino acids 34-60 of the human T-cell-specific surface glycoprotein CD28 precursor of GENBANK® Accession No. NP_001230007.1 or amino acids 151-177 of the mouse T-cell-specific surface glycoprotein CD28 precursor of GENBANK® Accession No. NP_031668.3 (SEQ ID NO: 15); or (iii) a sequence with at least 95% identity to amino acids 34-60 of the human T-cell-specific surface glycoprotein CD28 precursor of GENBANK® Accession No. NP_001230007.1 or amino acids 151-177 of the mouse T-cell-specific surface glycoprotein CD28 precursor of GENBANK® Accession No. NP_031668.3 (SEQ ID NO: 15).

In some embodiments of the presently disclosed CAR molecules, the binding domain is connected to the transmembrane domain by a hinge region. In some embodiments, the hinge region comprises amino acids of 101-158 of GENBANK® Accession No. AAA52771.1 (SEQ ID NO: 13), or a subsequence thereof.

In some embodiments, the presently disclosed CAR molecules further comprise a costimulatory domain, optionally a costimulatory domain comprising a functional signaling domain of a protein selected from the group consisting of OX40, CD2, CD3, CD27, CD28, CDS, ICAM-1, LFA-1 (CD11a/CD18), and 4-1BB (CD137), or optionally an amino acid sequence with at least 95% identity thereto.

In some embodiments, CAR molecules can further comprise an intracellular signaling domain, wherein in some embodiments the intracellular signaling domain comprises a functional signaling domain of 4-1BB, a functional signaling domain of CD3 zeta, or both, an amino acid sequence that has at least one, two, or three but not more than 10, or 5 modifications of said amino acid sequence, or an amino acid sequence at least 95% identical thereto.

In some embodiments, the presently disclosed CAR molecules further comprise a leader sequence, optionally a leader sequence comprising an amino acid sequence derived from amino acids 1-21 of the human CD8 alpha chain precursor (see e.g., GENBANK® Accession No. NP_001139345.1), or an amino acid sequence with at least 95% identity thereto.

In some embodiments, the presently disclosed nucleic acid molecules are present in a vector, optionally an expression vector. Thus, the presently disclosed subject matter provides in some embodiments vectors comprising the nucleic acid molecules of the presently disclosed subject matter and/or a nucleotide sequence encoding a presently disclosed CAR of the presently disclosed subject matter. In some embodiments, the vector is selected from the group consisting of a DNA vector, an RNA vector, a plasmid, a lentivirus vector, an adenovirus vector, an adeno-associated virus (AAV) vector, and a retrovirus vector, any of which can in some embodiments be an expression vector. In some embodiments, a vector of the presently disclosed subject matter further comprises a promoter, optionally an EF-1 promoter, operably linked to the nucleic acid molecule or the nucleotide sequence. In some embodiments, the vector is an in vitro transcribed vector. In some embodiments, the nucleic acid molecule or the nucleotide sequence further comprises and/or encodes a polyadenylation signal and/or a poly(A) tail. In some embodiments, the nucleic acid molecule or the nucleotide sequence in the vector further comprises a 3′-UTR.

Thus, in some embodiments the presently disclosed subject matter provides vectors comprising a nucleic acid sequence encoding a chimeric antigen receptor (CAR) of the presently disclosed subject matter, wherein the CAR comprises an antigen binding domain that binds to a TAA or an epitope thereof, a transmembrane domain, a costimulatory signaling domain of CD28, and a CD3 zeta signaling domain. In some embodiments, the antigen binding domain is an antibody or fragment thereof that binds to the TAA or the epitope thereof.

In some embodiments of the presently disclosed subject matter, the vectors are present within host cells. In some embodiments, the host cell is a human T cell, optionally a CD8⁺ T cell.

The presently disclosed subject matter also provides in some embodiments methods for making a cell expressing an anti-TAA CAR as disclosed herein. In some embodiments, the methods comprise transducing a T cell with a vector encoding an anti-TAA CAR as disclosed herein.

In some embodiments, the presently disclosed subject matter also provides methods for generating populations of RNA-engineered cells, which in some embodiments comprise introducing an in vitro transcribed RNA or synthetic RNA into a cell, where the RNA comprises a nucleic acid encoding an anti-TAA CAR molecule of the presently disclosed subject matter.

The presently disclosed subject matter also provides in some embodiments methods for expressing nucleic acids encoding CARs in vivo.

In some embodiments, the presently disclosed subject matter provides methods for generating a persisting population of genetically engineered T cells in a human or other mammal diagnosed with cancer. In some embodiments, the presently disclosed methods comprise administering to the human or other mammal a T cell genetically engineered to express a CAR that comprises an antigen binding domain that binds to a TAA or an epitope thereof, a transmembrane domain, a costimulatory signaling region comprising the CD28 signaling domain, and a CD3 zeta signaling domain, wherein the persisting population of genetically engineered T cells persists in the human for at least one month after administration. In some embodiments, the persisting population of genetically engineered T cells comprises at least one T cell that was administered to the human and/or a progeny cell thereof. In some embodiments, the persisting population of genetically engineered T cells comprises a memory T cell. In some embodiments, the persisting population of genetically engineered T cells persists in the human for at least three months, four months, five months, six months, seven months, eight months, nine months, ten months, eleven months, twelve months, two years, or at least three years after administration. In some embodiments, the cancer is a gastric-associated cancer, and in some embodiments the cancer is a cancer characterized by fibrosis.

The presently disclosed subject matter also provides in some embodiments methods for expanding a population of genetically engineered T cells in a human or other mammal diagnosed with cancer. In some embodiments, the methods comprise administering to the human or other mammal a T cell genetically engineered to express a CAR comprising an antigen binding domain that binds to a tTAA or an epitope thereof, a transmembrane domain, a costimulatory signaling region comprising the CD28 signaling domain, and a CD3 zeta signaling domain, wherein the administered genetically engineered T cell produces a population of progeny T cells in the human. In some embodiments, the progeny T cells in the human comprise a memory T cell. In some embodiments, the T cell administered to the human is an autologous T cell. In some embodiments, the population of progeny T cells persists in the human for at least three months, four months, five months, six months, seven months, eight months, nine months, ten months, eleven months, twelve months, two years, or at least three years after administration.

The presently disclosed subject matter also provides in some embodiments methods for modulating the amount of cytokine secreted by a T cell. In some embodiments, the methods comprise genetically engineering the T cell to express a CAR of the presently disclosed subject matter. In some embodiments, the amount of cytokine secreted by a T cell reduces the proliferation of T regulatory cells in vivo, in vitro, or ex vivo.

In some embodiments, the presently disclosed subject matter provides methods for reducing the amount of activation-induced calcium influx into a T cell. In some embodiments, the methods comprise genetically engineering the T cell to express a CAR of the presently disclosed subject matter. In some embodiments, reducing the amount of activation-induced calcium influx into a T cell prevents activation-induced cell death of the T cell in vivo, in vitro, or ex vivo.

### IV. Methods for Treatment and/or Prevention Employing CARs

The presently disclosed subject matter also provides methods for treating and/or preventing a disease, condition, or disorder associated with undesirable gastrin expression and/or biological activity, and/or with fibrosis, which in some embodiments can be fibrosis that inhibits access of a cell, tissue, or organ to the therapeutic compositions of the presently disclosed subject matter.

In some embodiments, the presently disclosed methods relate to providing an anti-tumor and/or anti-cancer treatment to a mammal, optionally a human. In some embodiments, the presently disclosed methods comprise administering to the mammal or the human an effective amount of a cell expressing a CAR molecule of the presently disclosed subject matter. The cell expressing the CAR molecule can be in some embodiments an autologous T cell and in some embodiments the cell is an allogeneic T cell.

The presently disclosed subject matter methods also relate to treating mammals, optionally humans, having a disease, condition, or disorder, wherein the methods comprise administering to the mammal an effective amount of a cell comprising a CAR molecule of the presently disclosed subject matter. In some embodiments, the disease, condition, or disorder is selected from a proliferative disease such as a cancer or malignancy or a precancerous condition such as a myelodysplasia, a myelodysplastic syndrome, or a preleukemia, or is a non-cancer related indication associated with aberrant expression of gastrin or aberrant gastrin signaling resulting from or associated with fibrosis. In some embodiments, the gastrin-associated disease, condition, or disorder is a tumor or a cancer that expresses gastrin or its receptor, such as but not limited to colon tumors and/or cancers, gastric tumors and/or cancers, pancreatic tumors and/or cancers, thyroid tumors and/or cancers, lung tumors and/or cancers, hepatocellular tumors and/or cancers, and esophageal tumors and/or cancers.

In some embodiments, the cells expressing the CAR molecule are administered as part of a combination therapy that also comprises administration of an agent that ameliorates one or more side effects associated with administration of the cell expressing the CAR molecule.

The presently disclosed subject matter also provides methods for treating a solid tumor in a human patient, optionally a solid tumor associated with a fibrotic tumor microenvironment. In some embodiments, the methods comprise administering to the human patient a pharmaceutical composition comprising an anti-tumor effective amount of a population of modified human T cells, optionally modified autologous T cells, wherein the T cells comprise a nucleic acid sequence that encodes a CAR of the presently disclosed subject matter. In some embodiments, the CAR comprises an antigen binding domain that binds to a TAA or an epitope thereof, optionally a hinge domain, a transmembrane domain, a CD28 costimulatory signaling region, and a CD3 zeta signaling domain. In some embodiments, the anti-tumor effective amount of T cells is 10⁴ to 10⁹ cells per kg body weight of the human patient. In some embodiments, the anti-tumor effective amount of T cells is 10⁵ to 10⁶ cells per kg body weight of the human patient. In some embodiments, the antigen binding domain is an antibody or a fragment thereof that binds to the TAA or the epitope thereof. In some embodiments, the antigen binding fragment comprises a Fab fragment or an scFv. In some embodiments, the modified T cells replicate in vivo in the human patient and/or form memory T cells in the human patient. In some embodiments, the modified T cells are administered intravenously to the human patient. In some embodiments, the modified T cells persist in the human patient, optionally for at least three, four, five, six, seven, eight, nine, ten, eleven, twelve, eighteen, twenty-four, thirty, or thirty-six months after administration.

The presently disclosed subject matter also relates in some embodiments to methods for stimulating T cell-mediated immune responses to a target cell population or tissue in a mammal, optionally a human. In some embodiments, the methods comprise administering to the mammal an effective amount of a cell genetically modified to express a CAR, wherein the CAR comprises an antigen binding domain that binds to a TAA or an epitope thereof, a transmembrane domain, a costimulatory signaling region comprising the CD28 signaling domain, and a CD3 zeta signaling domain.

In some embodiments, a method for inducing anti-tumor immunity in a mammal comprises administering to the mammal an effective amount of a cell genetically modified to express a CAR, wherein the CAR comprises an antigen binding domain that binds to a TAA or an epitope thereof, a transmembrane domain, a costimulatory signaling region comprising the CD28 signaling domain, and a CD3 zeta signaling, thereby inducing an anti-tumor immunity in the mammal.

The presently disclosed subject matter also relates in some embodiments to methods for treating a mammal having a disease, disorder, or condition associated with expression of a TAA or an epitope thereof. In some embodiments, the methods comprise administering to the mammal an effective amount of a cell genetically modified to express a CAR that comprises an antigen binding domain that binds to the TAA or an epitope thereof, a transmembrane domain, a costimulatory signaling region comprising the CD28 signaling domain, and a CD3 zeta signaling domain, thereby treating the mammal. In some embodiments, the cell is an autologous T cell.

The presently disclosed subject matter also relates in some embodiments to methods for treating a human with cancer. In some embodiments, the methods comprise administering to the human a T cell genetically engineered to express a CAR that comprises an antigen binding domain that binds to a TAA or an epitope thereof, a transmembrane domain, a costimulatory signaling region comprising the CD28 signaling domain, and a CD3 zeta signaling domain.

### V. Pharmaceutical Compositions

In some embodiments, the presently disclosed subject matter provides pharmaceutical compositions that in some embodiments can be employed in the methods of the presently disclosed subject matter.

As used herein, a “pharmaceutical composition” refers to a composition that is to be employed as part of a treatment or other method wherein the pharmaceutical composition will be administered to a subject in need thereof. In some embodiments, a subject in need thereof is a subject with a tumor and/or a cancer at least one symptom, characteristic, or consequence of which is expected to be ameliorated at least in part due to a biological activity of the pharmaceutical composition acting directly and/or indirectly on the tumor and/or the cancer and/or a cell associated therewith.

Techniques for preparing pharmaceutical compositions are known in the art, and in some embodiments pharmaceutical compositions are formulated based on the subject to which the pharmaceutical compositions are to be administered. For example, in some embodiments a pharmaceutical composition is formulated for use in a human subject. Thus, in some embodiments a pharmaceutical composition is pharmaceutically acceptable for use in a human.

The pharmaceutical compositions of the presently disclosed subject matter in some embodiments comprise a first agent that induces and/or provides an active and/or a passive humoral immune response against a gastrin peptide and/or a CCK-B receptor; and an immune checkpoint inhibitor (CPI). In some embodiments, the first agent is selected from the group consisting of a gastrin peptide, an anti-gastrin antibody, and an anti-CCK-R antibody. In some embodiments, the first agent comprises a gastrin peptide, optionally a gastrin peptide comprising, consisting essentially of, or consisting of an amino acid sequence selected from the group consisting of EGPWLEEEEE (SEQ ID NO: 1), EGPWLEEEE (SEQ ID NO: 2), EGPWLEEEEEAY (SEQ ID NO: 3), and EGPWLEEEEEAYGWMDF (SEQ ID NO: 4). In some embodiments, the glutamic acid residue an amino acid position 1 of any of SEQ ID NOs: 1-4 is a pyroglutamate residue. In some embodiments, the gastrin peptide is conjugated to an immunogenic carrier, optionally wherein the immunogenic carrier is selected from the group consisting of diphtheria toxoid, tetanus toxoid, keyhole limpet hemocyanin, and bovine serum albumin. In some embodiments, the gastrin peptide is conjugated to an immunogenic carrier via a linker, optionally wherein the linker comprises a ε-maleimido caproic acid N-hydroxysuccinamide ester.

In some embodiments, the linker and the gastrin peptide are separated by an amino acid spacer, optionally wherein the amino acid spacer is between 1 and 10 amino acids in length, further optionally wherein the amino acid spacer is 7 amino acids in length.

Pharmaceutical compositions of the presently disclosed subject matter that are designed to elicit humoral immune responses can in some embodiments further comprise an adjuvant, optionally an oil-based adjuvant. Exemplary adjuvants include but are not limited to montanide ISA-51 (Seppic, Inc.); QS-21 (Aquila Pharmaceuticals, Inc.); Arlacel A; oeleic acid; tetanus helper peptides; GM-CSF; cyclophosamide; bacillus Calmette-Guerin (BCG); corynbacterium parvum; levamisole, azimezone; isoprinisone; dinitrochlorobenezene (DNCB); keyhole limpet hemocyanins (KLH) including Freunds adjuvant (complete and incomplete); mineral gels; aluminum hydroxide (Alum); lysolecithin; pluronic polyols; polyanions; peptides; oil emulsions; nucleic acids (e.g., dsRNA) dinitrophenol; diphtheria toxin (DT); toll-like receptor (TLR, e.g., TLR3, TLR4, TLR7, TLR8 or TLR9) agonists (e.g, endotoxins such as lipopolysaccharide (LPS); monophosphoryl lipid A (MPL); polyinosinic-polycytidylic acid (poly-ICLC/HILTONOL®; Oncovir, Inc., Washington, DC, United States of America); IMO-2055, glucopyranosyl lipid A (GLA), QS-21—a saponin extracted from the bark of the Quillaja saponaria tree, also known as the soap bark tree or Soapbark; resiquimod (TLR7/8 agonist), CDX-1401—a fusion protein consisting of a fully human monoclonal antibody with specificity for the dendritic cell receptor DEC-205 linked to the NY-ESO-1 tumor antigen; Juvaris' Cationic Lipid-DNA Complex; Vaxfectin; and combinations thereof.

In some embodiments of the presently disclosed pharmaceutical compositions, the first agent comprises an amount of a gastrin peptide comprising, consisting essentially of, or consisting of an amino acid sequence selected from the group consisting of EGPWLEEEEE (SEQ ID NO: 1), EGPWLEEEE (SEQ ID NO: 2), EGPWLEEEEEAY (SEQ ID NO: 3), and EGPWLEEEEEAYGWMDF (SEQ ID NO: 4) effective to induce an anti-gastrin humoral response and the second agent comprises an amount of a immunotherapeutic molecule that is effective to induce or enhance a cellular immune response against a gastrin-associated tumor or cancer when administered to a subject who has gastrin-associated tumor or cancer.

In some embodiments of the presently disclosed pharmaceutical compositions, the first agent comprises one or more anti-CCK-B receptor antibodies and is present in the pharmaceutical composition in an amount sufficient to reduce or inhibit gastrin signaling via CCK-B receptors present on a gastrin-associated tumor or cancer when administered to a subject that has a gastrin-associated tumor or cancer.

The pharmaceutical compositions of the presently disclosed subject matter are in some embodiments employed to treat a gastrin-associated tumor and/or cancer. In some embodiments, pharmaceutical compositions of the presently disclosed subject matter are intended to treat pancreatic cancer.

Compositions as described herein comprise in some embodiments a composition that includes a pharmaceutically acceptable carrier. Suitable formulations include aqueous and non-aqueous sterile injection solutions that can contain antioxidants, buffers, bacteriostats, bactericidal antibiotics, and solutes that render the formulation isotonic with the bodily fluids of the intended recipient; and aqueous and non-aqueous sterile suspensions, which can include suspending agents and thickening agents. In some embodiments, a formulation of the presently disclosed subject matter comprises an adjuvant, optionally an oil-based adjuvant.

The compositions used in the methods can take such forms as suspensions, solutions, or emulsions in oily or aqueous vehicles, and can contain formulatory agents such as suspending, stabilizing, and/or dispersing agents. The compositions used in the methods can take forms including, but not limited to perioral, intravenous, intraperitoneal, intramuscular, and intratumoral formulations. Alternatively or in addition, the active ingredient can be in powder form for constitution with a suitable vehicle (e.g., sterile pyrogen-free water) before use.

The formulations can be presented in unit-dose or multi-dose containers, for example sealed ampules and vials, and can be stored in a frozen or freeze-dried (lyophilized) condition requiring only the addition of sterile liquid carrier immediately prior to use.

For oral administration, the compositions can take the form of, for example, tablets or capsules prepared by a conventional technique with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycollate); or wetting agents (e.g., sodium lauryl sulfate). The tablets can be coated by methods known in the art. For example, a neuroactive steroid can be formulated in combination with hydrochlorothiazide, and as a pH stabilized core having an enteric or delayed-release coating which protects the neuroactive steroid until it reaches the colon.

Liquid preparations for oral administration can take the form of, for example, solutions, syrups or suspensions, or they can be presented as a dry product for constitution with water or other suitable vehicle before use. Such liquid preparations can be prepared by conventional techniques with pharmaceutically acceptable additives such as suspending agents (e.g., sorbitol syrup, cellulose derivatives or hydrogenated edible fats); emulsifying agents (e.g. lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring, and sweetening agents as appropriate. Preparations for oral administration can be suitably formulated to give controlled release of the active compound. For buccal administration the compositions can take the form of tablets or lozenges formulated in conventional manner.

The compounds can also be formulated as a preparation for implantation or injection. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt).

The compounds can also be formulated in oils that are administered as water-in-oil emulsions, oil-in-water emulsions, or water-in-oil-in water emulsions.

The compounds can also be formulated in rectal compositions (e.g., suppositories or retention enemas containing conventional suppository bases such as cocoa butter or other glycerides), creams or lotions, or transdermal patches.

In some embodiments, the presently disclosed subject matter employs a composition that is pharmaceutically acceptable for use in humans. One of ordinary skill in the art understands the nature of those components that can be present in such a composition that is pharmaceutically acceptable for use in humans and also what components should be excluded from compositions that are pharmaceutically acceptable for use in humans.

As used herein, the phrases “treatment effective amount”, “therapeutically effective amount”, “treatment amount”, and “effective amount” are used interchangeably and refer to an amount of a therapeutic composition sufficient to produce a measurable response (e.g., a biologically or clinically relevant response in a subject being treated). Actual dosage levels of active ingredients in the pharmaceutical compositions of the presently disclosed subject matter can be varied so as to administer an amount of the active compound(s) that is effective to achieve the desired therapeutic response for a particular subject. The selected dosage level can depend upon the activity of the therapeutic composition, the route of administration, combination with other drugs or treatments, the severity of the condition being treated, the condition and prior medical history of the subject being treated, etc. However, it is within the skill of the art to start doses of the compound at levels lower than required to achieve the desired therapeutic effect and to gradually increase the dosage until the desired effect is achieved. The potency of a therapeutic composition can vary, and therefore a “therapeutically effective amount” can vary. However, one skilled in the art can readily assess the potency and efficacy of a candidate modulator of the presently disclosed subject matter and adjust the therapeutic regimen accordingly.

After review of the disclosure herein of the presently disclosed subject matter, one of ordinary skill in the art can tailor the dosages to an individual subject, taking into account the particular formulation, method of administration to be used with the composition, and other factors. Further calculations of dose can consider subject height and weight, severity and stage of symptoms, and the presence of additional deleterious physical conditions. Such adjustments or variations, as well as evaluation of when and how to make such adjustments or variations, are well known to those of ordinary skill in the art of medicine.

Thus, in some embodiments the term “effective amount” is used herein to refer to an amount of a composition comprising an agent that provides and/or induces a humoral or cellular immune response against a gastrin peptide and or comprising a nucleic acid that inhibits expression of a gastrin gene product, a pharmaceutically acceptable salt thereof, a derivative thereof, or a combination thereof sufficient to produce a measurable anti-tumor and/or anti-cancer biological activity. Actual dosage levels of active ingredients in composition of the presently disclosed subject matter can be varied so as to administer an amount of the active compound(s) that is effective to achieve the desired response for a particular subject and/or application. The selected dosage level can depend upon a variety of factors including the activity of the composition, formulation, route of administration, combination with other drugs or treatments, severity of the condition being treated, and physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are known to those of ordinary skill in the art.

For administration of a composition as disclosed herein, conventional methods of extrapolating human dosage based on doses administered to a murine animal model can be carried out using techniques known to one of ordinary skill in the art. Drug doses can also be given in milligrams per square meter of body surface area because this method rather than body weight achieves a good correlation to certain metabolic and excretionary functions. Moreover, body surface area can be used as a common denominator for drug dosage in adults and children as well as in different animal species as described by Freireich et al., 1966. Briefly, to express a mg/kg dose in any given species as the equivalent mg/m² dose, multiply the dose by the appropriate km factor. In an adult human, 100 mg/kg is equivalent to 100 mg/kg×37 kg/m²=3700 mg/m².

For additional guidance regarding formulations and doses, see U.S. Pat. Nos. 5,234,933; PCT International Publication No. WO 93/25521; Remington et al., 1975; Goodman et al., 1996; Berkow et al., 1997; Speight & Holdford, 1997; Ebadi, 1998; Duch et al., 1998; Katzung, 2001; Gerbino, 2005.

The presently disclosed compositions can be administered to a subject in any form and/or by any route of administration. In some embodiments, the formulation is a sustained release formulation, a controlled release formulation, or a formulation designed for both sustained and controlled release. As used herein, the term “sustained release” refers to release of an active agent such that an approximately constant amount of an active agent becomes available to the subject over time. The phrase “controlled release” is broader, referring to release of an active agent over time that might or might not be at a constant level. Particularly, “controlled release” encompasses situations and formulations where the active ingredient is not necessarily released at a constant rate, but can include increasing release over time, decreasing release over time, and/or constant release with one or more periods of increased release, decreased release, or combinations thereof. Thus, while “sustained release” is a form of “controlled release”, the latter also includes delivery modalities that employ changes in the amount of an active agent that are delivered at different times.

In some embodiments, the sustained release formulation, the controlled release formulation, or the combination thereof is selected from the group consisting of an oral formulation, a peroral formulation, a buccal formulation, an enteral formulation, a pulmonary formulation, a rectal formulation, a vaginal formulation, a nasal formulation, a lingual formulation, a sublingual formulation, an intravenous formulation, an intraarterial formulation, an intracardial formulation, an intramuscular formulation, an intraperitoneal formulation, a transdermal formulation, an intracranial formulation, an intracutaneous formulation, a subcutaneous formulation, an aerosolized formulation, an ocular formulation, an implantable formulation, a depot injection formulation, a transdermal formulation and combinations thereof. In some embodiments, the route of administration is selected from the group consisting of oral, peroral, buccal, enteral, pulmonary, rectal, vaginal, nasal, lingual, sublingual, intravenous, intraarterial, intracardial, intramuscular, intraperitoneal, transdermal, intracranial, intracutaneous, subcutaneous, ocular, via an implant, and via a depot injection. Where applicable, continuous infusion can enhance drug accumulation at a target site (see, e.g., U.S. Pat. No. 6,180,082). See also U.S. Pat. Nos. 3,598,122; 5,935,975; 6,106,856; 6,162,459; 6,495,605; and 6,582,724; and U.S. Patent Application Publication No. 2006/0188558 for transdermal formulations and methods of delivery of compositions. In some embodiments, the administering is via a route selected from the group consisting of peroral, intravenous, intraperitoneal, inhalation, and intratumoral.

The particular mode of administration of the compositions of the presently disclosed subject matter used in accordance with the methods disclosed herein can depend on various factors, including but not limited to the formulation employed, the severity of the condition to be treated, whether the active agents in the compositions (e.g., PAS) are intended to act locally or systemically, and mechanisms for metabolism or removal of the active agents following administration.

### VI. Methods and Uses

In some embodiments, the presently disclosed subject matter relates to employing pharmaceutical compositions in the context of various methods and/or uses related to treating gastrin-associated tumors and/or cancers, producing medicaments for treating gastrin-associated tumors and/or cancers, inhibiting growth of gastrin-associated tumors and/or cancers, inducing and/or enhancing humoral and/or cellular immune responses against gastrin-associated tumors and/or cancers, sensitizing tumors and/or cancers associated with gastrin and/or CCK-B receptor signaling in subjects to inducers of cellular immune responses directed against the tumors and/or cancers, preventing, reducing, and/or eliminating formation of fibrosis associated with tumors and/or cancers, particularly in the context of pancreatic cancer; preventing, reducing, and/or eliminating metastases of gastrin-associated tumors and/or cancers; increasing the number of tumor-infiltrating CD8⁺ lymphocytes in tumors and/or cancers; reducing the number of FoxP3⁺ inhibitory T-regulatory cells present in tumors and/or cancers; and increasing the number of T_{EMRA} cells in subject that respond to gastrin-associated tumors and/or cancers. Each of these methods and/or uses is described in more detail herein below.

VI.A. Methods for Treating Gastrin-associated Tumors and/or Cancers

In some embodiments, the presently disclosed subject matter relates to methods for treating gastrin-associated tumors and/or cancers. In some embodiments, the method comprises administering to a subject in need thereof (e.g., a subject with a gastrin-associated tumor and/or cancer) an effective amount of a composition that comprises a first agent that induces and/or provides an active and/or a passive humoral immune response against a gastrin peptide and/or a CCK-B receptor; and a second agent that induces and/or provides a cellular immune response against the gastrin-associated tumor or cancer. Thus, the presently disclosed methods in some embodiments rely on the use of pharmaceutical compositions that have one or more active agents that together provide two distinct immunotherapeutic activities: providing and/or inducing an active and/or a passive humoral immune response against a gastrin peptide and/or a CCK-B receptor, and inducing and/or providing a cellular immune response against the gastrin-associated tumor and/or cancer.

With respect to providing and/or inducing an active and/or a passive humoral immune response against a gastrin peptide and/or a CCK-B receptor, the first agent present in the pharmaceutical compositions of the presently disclosed subject matter is selected from the group consisting of a gastrin peptide designed to induce an active humoral response against gastrin, and/or an anti-gastrin antibody and/or an anti-CCK-R antibody designed to provide a passive humoral response against gastrin and/or a CCK-B receptor, in some embodiments a CCK-B receptor present on gastrin-associated tumor and/or cancer. While not wishing to be bound by any particular theory of action, the active and/or a passive humoral immune response against a gastrin peptide and/or a CCK-B receptor is designed to inhibit, either partially or completely, gastrin signaling in the gastrin-associated tumor and/or cancer via the CCK-B receptor by reducing gastrin binding to the CCK-B receptor by reducing the amount of circulating gastrin present in the subject and/or by interfering with gastrin binding to the CCK-B receptor with neutralizing and/or blocking antibodies.

Thus, in some embodiments the first agent comprises a gastrin peptide, optionally a gastrin peptide comprising, consisting essentially of, or consisting of an amino acid sequence selected from the group consisting of EGPWLEEEEE (SEQ ID NO: 1), EGPWLEEEE (SEQ ID NO: 2), EGPWLEEEEEAY (SEQ ID NO: 3), and EGPWLEEEEEAYGWMDF (SEQ ID NO: 4), wherein the glutamic acid residue at amino acid position 1 of any of SEQ ID NOs: 1-4 is a pyroglutamate residue. In some embodiments, the gastrin peptide is conjugated to an immunogenic carrier, optionally via a linker, further optionally a linker comprising a ε-maleimido caproic acid N-hydroxysuccinamide ester, in the pharmaceutical composition. Non-limiting examples of immunogenic carriers include diphtheria toxoid, tetanus toxoid, keyhole limpet hemocyanin, and bovine serum albumin. The structure of the first agent is described in more detail herein above, but in some embodiments the linker and the gastrin peptide are separated by an amino acid spacer, optionally wherein the amino acid spacer is between 1 and 10 amino acids in length, further optionally wherein the amino acid spacer is 7 amino acids in length.

As would be appreciated by one of ordinary skill in the art upon consideration of this disclosure, in some embodiments the pharmaceutical composition further comprises an adjuvant, optionally an oil-based adjuvant, to enhance the immunogenicity of the gastrin peptide and/or the gastrin peptide conjugate when an active anti-gastrin humoral immune response is desired.

In some embodiments, in order to induce a cellular immune response against the gastrin-associated tumor or cancer, the methods of the presently disclosed subject matter employ pharmaceutical compositions that comprise a CAR and/or a CAR-T cell, wherein the CAR binds to a TAA, a CAA, or an epitope thereof. Exemplary TAAs/CAAs include but are not limited to claudin18.2 (GENBANK® Accession No. NP_001002026.1; expressed by gastric and pancreatic cancers; see PCT International Patent Application Publication No. WO 2020/135674, U.S. Patent Application Publication No. 20180233511, and U.S. Pat. No. 10,377,822 for discussions of anti-claudin18.2 CARs), glypican-3 (GENBANK® Accession No. NP_001158089.1; expressed in liver cancer; see U.S. Patent Application Publication Nos. 2017/0369561 and 2019/0046659 for discussions of anti-glypican-3 CARs), mesothelin (GENBANK® Accession No. NP_005814.2; expressed in pancreatic cancer; see U.S. Pat. No. 9,272,002 and U.S. Patent Application Publication No. 2018/0244796 for discussions of anti-mesothelin CARs), carcinoembryonic antigen (CEA; GENBANK® Accession No. Q13982-1, expressed in gastric cancer, colon cancer, and liver metastases; see U.S. Patent Application Publication No. 2017/0145095 for a discussion of anti-CEA CARs), a prostate stem cell antigen (PSCA; GENBANK® Accession No. NP_005663.2, expressed in pancreatic cancer; see U.S. Patent Application Publication No. 2020/0140520 for a discussion of anti-PSCA CARs), and a CD70 antigen (GENBANK® Accession No. NP_001243.1, expressed by pancreatic cancer; see U.S. Patent Application Publication No. 2018/0230224 for a discussion of anti-CD70 CARs). CARs that bind to various TAAs and/or CAAs are commercially available and/or under development by various sources including CARsgen Therapeutics of hsanghai, China (e.g., CARs targeting claudin18.2 for gastric and pancreatic tumors and cancers), Kuur Therapeutics of Houston, Texas (e.g., CARs targeting glypican3 for liver tumors and cancers), the University of Pennsylvania (e.g., CARs targeting mesothelin for pancreatic tumors and cancers), Sorrento Therapeutics, Inc., of San Diego, California (e.g., CARs targeting CEA for gastric, colon, and pancreatic tumors and cancers and for liver metastases), Bellicum Pharmaceuticals of Houston, Texas (e.g., CARs targeting PSCA for pancreatic tumors and cancers), and the National Cancer Institute (NCI; e.g., CARs targeting CD70 pancreatic tumors and cancers).

In some embodiments, the compositions and methods of the presently disclosed subject matter can employ one or more checkpoint inhibitors. As is known, checkpoint inhibitors inhibit one or more biological activities of target polypeptides that have immune checkpoint activities. Exemplary such polypeptides include cytotoxic T-lymphocyte antigen 4 (CTLA4) polypeptides, programmed cell death-1 receptor (PD-1) polypeptides, and programmed cell death 1 receptor ligand (PD-L1) polypeptides. In some embodiments, a checkpoint inhibitor comprises an antibody or a small molecule that binds to and/or interferes with interactions between T cells and tumor cells by inhibiting or preventing interactions between PD-1 polypeptides and PD-L1 polypeptides. Exemplary such antibodies and small molecules include but are not limited to Ipilimumab, Tremelimumab, Nivolumab, Pidilizumab, Pembrolizumab, AMPS 14, AUNP12, BMS-936559/MDX-1105, Atezolizumab, MPDL3280A, RG7446, R05541267, MEDI4736, Avelumab and Durvalumab.

The pharmaceutical compositions of the presently disclosed subject matter can include various amounts of each active agent, provided that both humoral and cellular responses are induced and/or provided in the subject, and the amounts of each agent present in the pharmaceutical compositions can be adjusted in order to maximize the effectiveness of the treatment and/or minimize undesirable side effects thereof. However, in some embodiments a pharmaceutical composition of the presently disclosed subject matter is administered in a dose selected from the group consisting of about 50 μg to about 1000 about 50 μg to about 500 about 100 μg to about 1000 about 200 μg to about 1000 and about 250 μg to about 500 and optionally wherein the dose is repeated once, twice, or three times, optionally wherein the second dose is administered 1 week after the first dose and the third dose, if administered, is administered 1 or 2 weeks after the second dose.

In some embodiments, a method for treating a gastrin-associated tumor and/or cancer of the presently disclosed subject matter comprises administering to a subject in need thereof a first agent that directly or indirectly inhibits one or more biological activities of gastrin in the tumor and/or cancer and a second agent comprising a stimulator of a cellular immune response against the tumor and/or the cancer. As such, in some embodiments the first agent directly or indirectly inhibits one or more biological activities of gastrin in the tumor and/or cancer by providing and/or inducing a humoral immune response against a gastrin peptide, optionally wherein the agent is selected from the group consisting of an anti-gastrin antibody and a gastrin peptide that induces production of neutralizing anti-gastrin antibodies in the subject; and/or comprises a nucleic acid that inhibits expression of a gastrin gene product. Nucleic acids that inhibit expression of a gastrin gene product would be understood by one of ordinary skill in the art after consideration of this disclosure, and examples are discussed herein above. In some embodiments, the first agent enhances access of the tumor and/or the cancer to the second agent, which in some embodiments can be an anti-tumor and/or anti-cancer immunotherapeutic molecule such as but not limited to a CAR that binds to a TAA and/or a CAA and/or is a CAR-T cell comprising an anti-tumor and/or anti-cancer CAR.

Anti-gastrin antibodies are known in the art and are described in U.S. Pat. Nos. 5,607,676; 5,609,870; 5,622,702; 5,785,970; 5,866,128; and 6,861,510. See also PCT International Patent Application Publication Nos. WO 2003/005955 and WO 2005/095459. The content of each of these U.S. Patents and PCT International Patent Application Publications is incorporated herein in its entirety. In some embodiments, an anti-gastrin antibody is an antibody directed against an epitope present within gastrin-17 (G17). In some embodiments, the epitope is present within one or more of the amino acid sequences EGPWLEEEEE (SEQ ID NO: 1), EGPWLEEEE (SEQ ID NO: 2), EGPWLEEEEEAY (SEQ ID NO: 3), and EGPWLEEEEEAYGWMDF (SEQ ID NO: 4).

In some embodiments, administration of a pharmaceutical composition of the presently disclosed subject matter to a subject induces a reduction in and/or prevents the development of fibrosis associated with the pancreatic cancer.

In some embodiments, the presently disclosed treatment methods are designed to inhibit growth and/or survival of a gastrin-associated tumor and/or cancer in a subject. In some embodiments, the presently disclosed methods thus comprise administering to the subject a composition that comprises a first agent comprising a gastrin immunogen, one or more anti-gastrin antibodies, one or more anti-CCK-B receptor antibodies, or any combination thereof; and a second agent comprising a checkpoint inhibitor.

Thus, in some embodiments the presently disclosed subject matter provides uses of the pharmaceutical compositions disclosed herein for the preparation of medicaments to treat gastrin-associated tumors and/or cancers as well as uses of the pharmaceutical compositions disclosed herein to treat gastrin-associated tumors and/or cancers.

In some embodiments, the multi-agent pharmaceutical compositions disclosed herein provide enhanced, more efficacious, and/or more successful treatment of gastrin-associated tumors and/or cancers than would treating a similar subject with the any of the agents individually.

VI.B. Methods for Inducing and/or Enhancing Access of Immunotherapeutic Molecules to Gastrin-associated Tumors and/or Cancers

PAS has been shown to alter the tumor microenvironment (TME) rendering it more susceptible to immune-based therapy. PAS therapy reduces fibrosis, repolarizes tumor associated macrophages from tumor-promoting (M2) to tumor killing (M1) macrophages, and increases the number of tumor infiltrating lymphocytes (CD8⁺). It has been proposed that the lack of efficacy of CAR T cells in solid tumors in related to these features of the TME impeding the influx of CART cells. We believe the addition of PAS will improve the penetration of the CAR T cells and their efficacy in treating those with solid tumors. We propose to test the role of PAS in enhancing CAR T cell therapies in GI cancers with an emphasis on pancreatic cancer.

Thus, in some embodiments the presently disclosed subject matter also provides methods for inducing and/or enhancing access of immunotherapeutic molecules to gastrin-associated tumors and/or cancers in subject. In some embodiments, the methods comprise administering to a subject that has a gastrin-associated tumor or cancer an effective amount of a composition comprising an agent that reduces or inhibits gastrin signaling via CCK-B receptors present on a gastrin-associated tumor or cancer, thereby enhancing access of immunotherapeutic molecules to the subject's gastrin-associated tumor and/or cancer.

As used herein, the phrase “immunotherapeutic molecule” refers to any molecule that produces and/or contributes to an immune response sufficient to achieve at least one therapeutic effect in an individual. In some embodiments, an immunotherapeutic molecule is a molecule that binds to a tumor- and/or cancer-associated antigen and results in an immune response to the tumor- and/or cancer-associated antigen. In some embodiments, the immunotherapeutic molecule is a chimeric antigen receptor (CAR), which in some embodiments is present on the surface of a CAR T cell.

As used herein, the phrase “inducing and/or enhancing a cellular immune response against a gastrin-associated tumor and/or cancer” and grammatical variants of refers to a circumstance where as a result of administering to a subject that has a gastrin-associated tumor or cancer an effective amount of a composition comprising an agent that reduces or inhibits gastrin signaling via CCK-B receptors present on a gastrin-associated tumor or cancer, a level of a T cell-based immune response is higher in the subject at a relevant time post-administration than would have been present in the subject in the absence of the treatment. Agents that reduce or inhibit gastrin signaling via CCK-B receptors present on a gastrin-associated tumor or cancer include the agents disclosed herein that can interfere with an interaction of a gastrin peptide and a CCK-B receptor, and include but are not limited to gastrin peptides and/or immunogens, anti-gastrin antibodies, anti-CCK-B receptor antibodies, small molecule inhibitors of gastrin/CCK-B signaling, and combinations thereof.

In some embodiments, the presently disclosed subject matter also provides methods for sensitizing tumors and/or cancers associated with gastrin and/or CCK-B receptor signaling in a subject to inducers of cellular immune responses directed against the tumors and/or cancers. As used herein, the phrase “sensitizing tumors and/or cancers associated with gastrin and/or CCK-B receptor signaling in a subject to inducers of cellular immune responses” refers to treatments that result in levels of cellular immune responses in subjects when one or more inducers of a cellular immune response is administered to the subject as compared to levels of cellular immune responses in subjects when one or more inducers of a cellular immune response is administered to the subject in the absence of the treatment. In some embodiments, an inducer of a cellular immune response to a tumor and/or a cancer is a CAR, optionally wherein the CAR is present on a CAR-T cell.

In some embodiments, the methods comprise administering to a subject a composition comprising a first agent that induces and/or provides an active and/or a passive humoral immune response against a gastrin peptide, and a second agent that induces and/or provides a cellular immune response against the tumor and/or the cancer, or a combination thereof, optionally wherein the first agent and the second agent are individually selected from the group consisting of a gastrin peptide and/or a fragment and/or a derivative thereof that induces a cellular immune response or production of neutralizing anti-gastrin antibodies in the subject and a neutralizing anti-gastrin antibody and/or a fragment and/or derivative thereof and; and/or a composition comprising a nucleic acid that inhibits expression of a gastrin gene product; and/or a composition comprising an agent that blocks the biological function of gastrin at the CCK-B receptor. In some embodiments, the anti-gastrin antibody is an antibody directed against an epitope present within gastrin-17 (G17).

Accordingly, in some embodiments the instant methods for sensitizing tumors and/or cancers associated with gastrin and/or CCK-B receptor signaling in a subject to inducers of cellular immune responses comprises administering to the subject a pharmaceutical composition as disclosed herein in order to induce and/or provide to the subject both an active and/or a passive humoral immune response against a gastrin peptide in the subject as well as to induce and/or provide a cellular immune response against the tumor and/or the cancer.

VI.C. Methods for Preventing, Reducing, and/or Eliminating Fibrosis Associated with Tumors and/or Cancers

Various tumors and cancers, including but not limited to solid tumors, pancreatic cancer, etc., are also characterized by a dense fibrotic environment (Neesse et al., 2011), which helps promote angiogenesis and creates a physical barrier that could inhibit the penetration of immunotherapeutics to the tumor site (Templeton & Brentnall, 2013). Disclosed herein is the unexpected and surprising observation that that with PAS administration, optionally in combination with one or more other anti-tumor and/or anti-cancer agents including but not limited to immune checkpoint inhibitors, the fibrotic nature that is a hallmark of the solid tumor (e.g., the pancreatic cancer) can be reduced. While not wishing to be bound by any particular theory of operation, a reduction in fibrosis can facilitate greater penetration of other active agents, including but not limited to macromolecules like CARs, CAR-T cells, checkpoint mAbs, etc. This could at least partially explain why certain solid tumors and other cancers have to date been characterized by relative insensitivity to treatment with check point inhibitors and other anti-tumor/anti-cancer therapeutic modalities, perhaps due to lack of penetration of the immunotherapeutic molecules and/or checkpoint mAbs to the tumor cells. Therefore, an aspect of the presently disclosed subject matter is that PAS in combination with other anti-tumor/antio-cancer molecules (e.g., CARs, immune checkpoint inhibitors, etc.) have anti-tumor/anti-cancer activity separately when given as monotherapy, but when given as a combination therapy as disclosed herein, they have much greater activity.

Novel and innovative drug combinations with diverse but complementary or even synergistic mechanisms of action are provided in accordance with the presently disclosed subject matter to address the inherently fibrotic nature of certain tumors and cancers and to be beneficial to allow greater access to the tumor environment of large therapeutic molecules (such as but not limited to monoclonal antibodies (mAbs), CARs, etc). While not wishing to be bound by any particular theory of operation, PAS plus CARs (optionally in combination with one or more immune checkpoint inhibitors) when administered together as part of a combination therapy can provide a synergistic effect to make tumors and cancers more accessible to chemotherapeutics such as CAR-T cells and/or immune checkpoint inhibitor drugs by reducing the fibrosis associated with the tumors and/or cancers, thereby allowing anti-tumor/anti-cancer therapeutics to induce a cellular immune response (e.g., anti-tumor CAR-T cell) against a gastrin-associated tumor.

Treatment with PAS results in a humoral immunological response (i.e., an antibody response) to the autocrine and paracrine tumor/cancer growth factor gastrin. In so doing, PAS affects the tumor/cancer phenotype by affecting cell proliferation, apoptosis, angiogenesis, invasion, and metastasis. As disclosed herein, PAS is also effective in decreasing fibrosis associated with solid tumors such as but not limited to PDAC. While not wishing to be bound by any particular theory of operation, this is believed to enhance the ability of large molecules, such as but not limited to immunotherapeutic molecules (e.g., CARs and immune checkpoint inhibitory mAbs), to gain greater access to the tumor site, which in turn would be expected to promote a much greater cellular immune effect. PAS also results in a cellular immune response to gastrin. Thus, disclosed herein are methods for treating tumors and/or cancers by PAS administration in conjunction with the administration of CARs, CAR-T cells, and/or immune checkpoint inhibitors such as anti-PD-1, anti-PD-L1, and/or anti-CTLA-4 mAbs to address the inherent fibrotic as well as recalcitrant nature of certain tumors and cancers in resistance to therapeutic agents that need access to the tumor and/or cancer for efficacy.

Therefore, in some embodiments the presently disclosed subject matter provides methods for preventing, reducing, and/or eliminating formation of fibrosis associated with a tumor and/or a cancer, optionally pancreatic cancer and/or a solid tumor, by contacting cells of the tumor and/or the cancer with an agent that directly or indirectly inhibits one or more biological activities of gastrin in the tumor and/or cancer. Agents that directly or indirectly inhibit one or more biological activities of gastrin are disclosed herein above, and include agents that provide and/or induce humoral immune responses against gastrin peptides (such as but not limited to anti-gastrin antibodies, and/or fragments and/or derivatives thereof), and gastrin peptides that induce production of neutralizing anti-gastrin antibodies in the subject; inhibitory nucleic acids that inhibit expression of gastrin gene products; small molecule compounds that block the function of the gastrin hormone, and any combination thereof. In some embodiments, the anti-gastrin antibodies comprise an antibody directed against an epitope present within gastrin-17 (G17), which epitope is in some embodiments present within one or more of the amino acid sequences EGPWLEEEEE (SEQ ID NO: 1), EGPWLEEEE (SEQ ID NO: 2), EGPWLEEEEEAY (SEQ ID NO: 3), and EGPWLEEEEEAYGWMDF (SEQ ID NO: 4).

As with other immunogenic forms of gastrin and gastrin peptides disclosed herein, in some embodiments the gastrin peptides are conjugated to an immunogenic carrier, optionally an immunogenic carrier selected from the group consisting of diphtheria toxoid, tetanus toxoid, keyhole limpet hemocyanin, and bovine serum albumin.

In some embodiments, the methods for preventing, reducing, and/or eliminating formation of fibrosis associated with a tumor and/or a cancer, optionally solid tumors and/or pancreatic cancer further comprise contacting the tumor and/or the cancer with a second agent comprising a stimulator of a cellular immune response against the tumor and/or the cancer such as but not limited to an immunotherapeutic agent such as a CAR or a CAR-T cell. Other exemplary stimulators of cellular immune responses that can be employed in conjunction with the first agent and/or the immunotherapeutic agents include immune checkpoint inhibitors such as those that inhibit a biological activity of a target polypeptide selected from the group consisting of cytotoxic T-lymphocyte antigen 4 (CTLA4), programmed cell death-1 receptor (PD-1), and programmed cell death 1 receptor ligand (PD-L1), including but not limited to Ipilimumab, Tremelimumab, Nivolumab, Pidilizumab, Pembrolizumab, AMPS 14, AUNP12, BMS-936559/MDX-1105, Atezolizumab, MPDL3280A, RG7446, R05541267, MEDI4736, and Avelumab.

In some embodiments, the tumor and/or cancer for which preventing, reducing, and/or eliminating the formation of fibrosis therein is a solid tumor or pancreatic cancer.

Summarily, in some embodiments the presently disclosed subject matter relates to uses of the presently disclosed compositions comprising gastrin immunogens in conjunction with immunotherapeutic agents (e.g., CARs and/or CAR-T cells, optionally in combination with one or more immune checkpoint inhibitors) to treat gastrin-associated tumors and/or cancers, either alone as a front-line therapy, in combination with other front-line therapies, or in combination with any other therapy that would be appropriate for a subject who has a gastrin-associated tumor and/or cancer.

VI.D. Methods for Sensitizing a Solid Tumor and/or Cancer to a Chimeric Antigen Receptor-T (CAR-T) Cell Therapy

In some embodiments, the presently disclosed subject matter also provides methods for sensitizing solid tumors and/or cancers in subjects to a chimeric antigen receptor-T (CAR-T) cell therapy. As used herein, the phrase “sensitizing a solid tumor and/or cancer to a chimeric antigen receptor-T (CAR-T) cell therapy” refers to a treatment that results in a solid tumor and/or cancer becoming more susceptible to a CAR-T cell therapy as a result of the treatment as compared to the susceptibility of the solid tumor and/or cancer to the CAR-T cell therapy than it would have been in the absence of the treatment. While not wishing to be bound by any particular theory of operation, in some embodiments the treatment results in greater accessibility of the tumor to the CAR-T cell therapy, which in some embodiments can comprise reducing fibrosis or some other physical impediment to the CAR-T cells entering the solid tumor.

Thus, in some embodiments the methods comprise administering to the subject a first composition comprising, consisting essentially of, or consisting of a conjugate comprising, consisting essentially of, or consisting of a gastrin immunogen conjugated to an immunogenic carrier, optionally conjugated via a linker, in an amount and via a route sufficient to enhance entry of the CAR-T cell into the solid tumor and/or cancer; and administering to the subject a second composition comprising, consisting essentially of, or consisting of a CAR-T cell that is targeted against an antigen present within the solid tumor and/or cancer, whereby the solid tumor and/or cancer in the subject is sensitized to the CAR-T cell therapy. As set forth herein, in some embodiments the solid tumor and/or the cancer is a solid gastrointestinal tumor and/or cancer, optionally a solid gastrin-dependent gastrointestinal tumor and/or cancer, further optionally a solid pancreatic tumor and/or cancer.

The presently disclosed methods can also be employed in the context of a combination therapy, wherein in some embodiments the methods further comprise administering to the subject one or more additional anti-tumor and/or anti-cancer therapies. The one or more additional anti-tumor and/or anti-cancer therapies can be additional anti-tumor and/or anti-cancer therapies appropriate for the type of tumor and/or cancer that is being treated with the CAR-T cell therapy. As such, the one or more additional anti-tumor and/or anti-cancer therapies can in some embodiments comprise, consist essentially of, or consist of administering to the subject an immune checkpoint inhibitor (CPI) such as but not limited to a cytotoxic T-lymphocyte antigen 4 (CTLA4) inhibitor, programmed cell death-1 receptor (PD-1) inhibitor, a programmed cell death 1 receptor ligand (PD-L1) inhibitor, or any combination thereof. Exemplary CPIs include, but are not limited to Ipilimumab, Tremelimumab, Nivolumab, Pidilizumab, Pembrolizumab, AMP514, AUNP12, BMS-936559/MDX-1105, Atezolizumab, MPDL3280A, RG7446, R05541267, MEDI4736, and Avelumab.

As a result of sensitizing a solid tumor and/or cancer in a subject to a chimeric antigen receptor-T (CAR-T) cell therapy, the presently disclosed method reduces and/or inhibits growth of the tumor and/or the cancer in the subject as compared to what would have occurred had the first composition not been administered.

### VII. Conclusion

The presently disclosed subject matter thus relates in some embodiments to combination therapies for the treatment of cancer using a combination of methods that individually or together generate both a humoral antibody immune response (using, for example, the gastrin cancer vaccine PAS) and a cellular T cell immune response (using, for example, the gastrin cancer vaccine PAS, a CAR that binds to a TAA and/or a CAA, optionally a CAR that is present on a CAR-T cell, further optionally in combination with an immune checkpoint inhibitor). More particularly, unexpected additive and/or synergistic efficacies in treating human and animal gastrointestinal tumors using the instantly described combination of drug classes that generate humoral and cellular immune anti-tumor responses in combination with cellular immune anti-tumor effects are described.

More particularly, the presently disclosed subject matter relates in some embodiments to using specific combinations of drugs that (i) induce humoral B cell immune responses to a tumor growth factor or circulating tumor growth factor (e.g., a gastrin immunogen such as but not limited to POAS; and (ii) induce and/or enhance cellular immune responses (i.e., anti-tumor and/or cancer T cell responses) directed against the tumor and/or cancer to elicit a cytotoxic T lymphocyte response (e.g., using a CARs and/or CAR-T cells, optionally in combination with one or more CPIs).

As such, in some embodiments disclosed herein are methods for treating human and animal tumors and cancers using a combination of a gastrin cancer vaccine in combination with a second active agent that overcomes tumor immunity, particularly tumor immunity directed towards anti-tumor T cell immune responses, and optionally one or more further actives that overcome immune checkpoint failure. Thus, in some embodiments the presently disclosed subject matter relates to treating specific human cancers with a cancer vaccine directed at eliciting a B cell and/or antibody immune response and a cellular immune response to the active form of the growth factor gastrin and/or another TAA and/or CAA, with the unexpected observation that this vaccine treatment also results in making the tumor more responsive to treatment with CARs, CAR-T cells, and in some embodiments also more responsive to immune checkpoint inhibitors, thus creating an unexpected, additive, or even synergistic combination therapeutic effect that enhanced anti-tumor efficacy.

Additionally, the pharmaceutical compositions of the presently disclosed subject matter can be employed for preventing, reducing, and/or eliminating metastasis of a gastrin-associated tumor or cancer by administering to a subject having a gastrin-associated tumor or cancer an amount of a pharmaceutical composition as disclosed herein sufficient to enhance the number of CD8⁺ tumor infiltrating lymphocytes (TILs), which in some embodiments are CAR-T cells comprising a CAR that binds to a TAA and/or a CAA expressed by the tumor and/or cancer to be treated.

With respect to the compositions and methods of the presently disclosed subject matter, in some embodiments the administering results in improves survival of the subject, reduced tumor growth, and/or enhanced efficacy of a chemotherapeutic agent and/or an immune checkpoint therapy in the subject as compared to that which would have occurred had the pharmaceutical composition not been administered.

### EXAMPLES

### Materials and Methods for the Examples

The following EXAMPLES provide illustrative embodiments. In light of the present disclosure and the general level of skill in the art, those of skill will appreciate that the following EXAMPLES are intended to be exemplary only and that numerous changes, modifications, and alterations can be employed without departing from the scope of the presently disclosed subject matter.

Cell Lines. Two murine cell lines were evaluated in this investigation. Murine gastric cancer cell NCC-S1 (NCC; Park et al., 2015) was provided by Dr. Kim through his collaborator Dr. Timothy Wang of Columbia University (New York, New York, United States of America). These gastric cancer cells have been shown to be metastatic when grown orthotopically in syngeneic mouse models. The second gastric cancer cell line YTN-16 (YTN), was established and provided by Professor Sachiyo Nomura (Yamamoto et al., 2018) through her collaborator Dr. James R. Goldenring of Vanderbilt University School of Medicine (Nashville, Tennessee, United States of America). The YTN cells are known to be invasive and metastatic even after subcutaneous injection in mice. Before the cells were used in animals, they were tested by IMPACT II PCR Profile and were negative for all pathogens. YTN cells were grown in culture using DMEM and NCC cells were grown in RPMI media; both with 10% fetal bovine serum in a humidified 5% CO₂ incubator at 37° C.

Cell line and receptor characterization by Real-Time PCR. Total RNA was extracted from cells (Qiagen) and subjected to qRT-PCR in the Fast cycling mode using a thermal cycler (Applied Biosystems) to examine the expression of the cholecystokinin B receptor (CCK-BR) and PD-L1 expression. Primers used included the following:

(SEQ ID NO: 7)

| | |
|---|---|
| CCK-BR Forward: | 5′-GATGGCTGCTACGTGCAACT-3′ |

(SEQ ID NO: 8)

| | |
|---|---|
| CCK-BR Reverse: | 5′-CGCACCACCCGCTTCTTAG-3′ |

(SEQ ID NO: 9)

| | |
|---|---|
| PD-L1 Forward: | 5′-TGCGGACTACAAGCGAATCACG-3′ |

(SEQ ID NO: 10)

| | |
|---|---|
| PD-L1 Reverse: | 5′-CTCAGCTTCTGGATAACCCTCG-3′ |

qRT-PCR was set to the Fast cycling mode (primer Tm>60° C.). HPRT was used as a normalizer control gene. Control RNA was extracted from mouse liver because it does not express either CCK-BR or PD-L1.

In Vivo Animal Studies. All animal studies were done in an ethical fashion and under the approval of the Institutional Animal Care and Use Committee (IACUC) of Georgetown University (Washington D.C., United States of America). Several attempts were made to establish tumors in C57BL/6 mice using the NCC cells. The first attempt included the injection of luciferase tagged 5×10⁵ NCC cancer cells orthotopically into the stomach subserosa (n=40). After imaging with luciferin and dissecting mice, no tumors were found. The NCC cells were then injected subcutaneously on the right flank with a total of 0.1 mL volume of 1.5×10⁶ NCC cancer cells, but after 33 days, no tumors formed. YTN (5×10⁶) cells were injected into each of 40 female C57BL/6 mice in 0.2 mL volume. The YTN cells are known to be invasive and metastatic even after subcutaneous injection in mice (Yamamoto et al., 2018). Tumor growth was measured weekly with calipers and volume calculated by L×W2×0.5.

Treatments. Mice were divided into four treatment groups (n=10 each). Control mice were treated with PBS in 0.1 mL i.p. injection given at the same time as the other treatments. PD-1 antibody 50 μg i.p. (PD-1 Ab; Clone RMPI-14 was purchased from Bio X Cell, West Lebanon, New Hampshire, United States of America) was administered at baseline (one week after tumor inoculation; week 0) and at week 1, 3, and 6. 250 μg PAS was administered subcutaneously in 0.1 mL volume at the same time as PD-1 Ab and also at week 9. After 10-weeks of growth the control mice were appearing moribund and the mice were ethically euthanized, tumors removed and weighed and metastases counted.

Tissue Analysis. All observed metastases counted were dissected and formalin fixed and paraffin embedded for confirmation by hematoxylin and eosin (H&E) staining. Tumors were reacted with Masson's trichrome stain for analysis of fibrosis in the tumor microenvironment. To determine the proliferation index of the tumors, tissue sections (5 μm) were reacted with a rabbit monoclonal antibody for Ki67 (Catalog No. CRM325; Biocare Medical, Pacheco, California, United States of America; 1:80 dilution). Immunohistochemical staining was also performed of tumor tissue sections (5 μm) to evaluate tumor infiltrating lymphocytes with CD8, (Catalog No. 98941; Cell Signaling Technology, Danvers, Massachusetts, United States of America; 1:25 dilution) and with rabbit polyclonal antibody against arginase-1 (Catalog No. PAS-29645; ThermoFisher Scientific, Waltham, Massachusetts, United States of America; 1:1,800 dilution) to examine M2-polarized tumor associated macrophages.

Statistical analysis. Tumor growth rates were analyzed using linear regression analysis to compare slopes of the growth curves between each treatment group. Slides were scanned using an Aperio GT450 machine and images analyzed with software from Aperio Image Scope for the number of immunoreactive cells per high powered field (for Ki67 and CD8 cells). Images at the same magnification and identical surface area were taken (up to N=10 per slide) for each tumor using the Aperio software and fibrosis and M2 polarized macrophage integrative density was analyzed with ImageJ computer software. Raw data results from images were analyzed using ANOVA and T-Test (with Bonferroni correction for multiple comparisons to controls) with GraphPad Prism version 9.

### Example 1

### Enhancement of Tumor Accessibility to Immunotherapy

A subject is identified with a solid tumor that is refractory to anti-tumor immunotherapy, potentially because anti-tumor immunotherapeutics cannot gain access to the tumor microenvironment, optionally because of the presence of fibrosis in the solid tumor. The subject is administered a first composition comprising, consisting essentially of, or consisting of a conjugate comprising, consisting essentially of, or consisting of a gastrin immunogen conjugated to an immunogenic carrier, optionally conjugated via a linker, in an amount and via a route sufficient to enhance anti-tumor T cell entry into the tumor. The administration of the first composition is repeated, as necessary, one or more times until the fibrosis is reduced to a degree sufficient to increase access of a CAR and/or a CAR-T cell to the tumor. A second composition comprising, consisting essentially of, or consisting of a CAR and/or one or more CAR-T cells that bind to a TAA present on the solid tumor is then administered to the subject on one or more occasions in an amount and via a route sufficient to permit access of the tumor to the CAR and/or the CAR-T cell, such that an immune response against the tumor is induced in the subject, thereby treating the solid tumor in the subject. Additional administrations of the first composition and/or the second composition are performed as needed to treat the solid tumor in the subject.

### Example 2

### Characterization of Gastric Cancer Cells for CCK-BR and PD-L1 Receptors

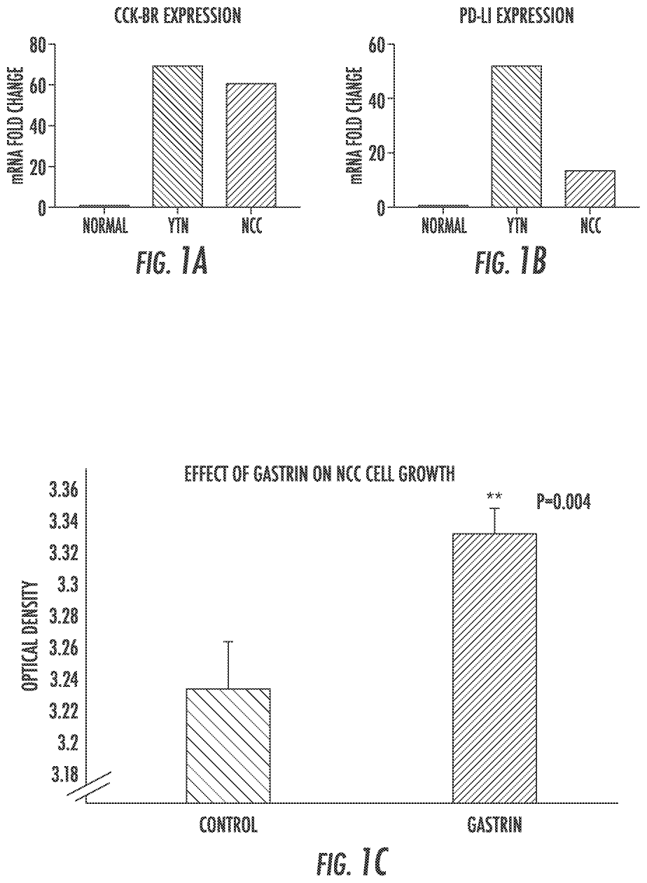

Two separate murine gastric cancer cells were evaluated for expression of CCK-BR, PD-L1 receptors and gastrin peptide in vitro. Gene expression of CCK-BR and PD-L1 were increased in both NCC and YTN gastric cancer cells compared to noncancerous mouse tissues (FIG. 1). CCK-BR expression was increased greater than 60-fold in mouse YTN and NCC gastric cancer cells compared to normal mouse tissues (FIG. 1A). PD-L1 mRNA expression was increased 52-fold in YTN cells and 24-fold in NCC cells over normal tissues (FIG. 1B). Growth of NCC cells increased significantly (p=0.004) when exposed to exogenous gastrin (FIG. 1C). Immunocytochemistry revealed endogenous gastrin peptide expression in both NCC (FIG. 1D) and YTN (FIG. 1E) gastric cancer cells suggesting that these gastric cancer cells produce their own gastrin peptide to stimulate growth via the CCK-BR in an autocrine fashion. Control cells reacted with the secondary antibody alone were negative for staining (FIG. 1F).

### Example 3

### Effects of PAS and PD-1 on Growth and Metastases of YTN Tumors

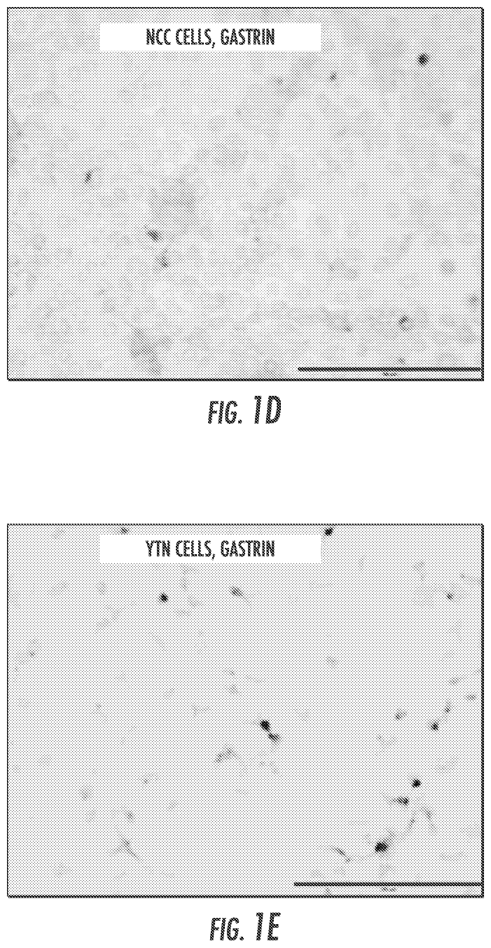

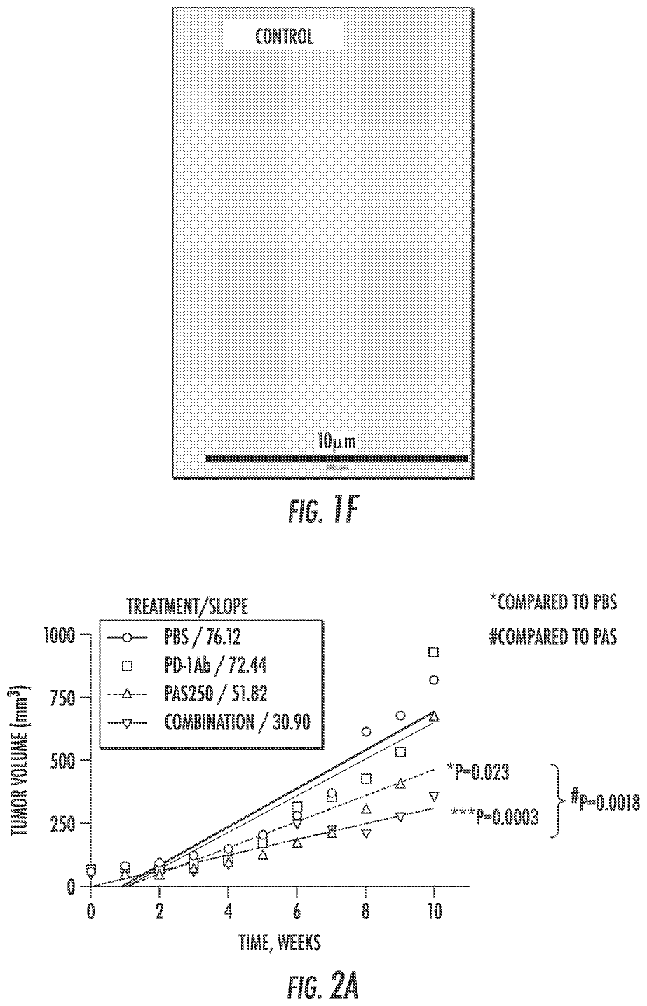

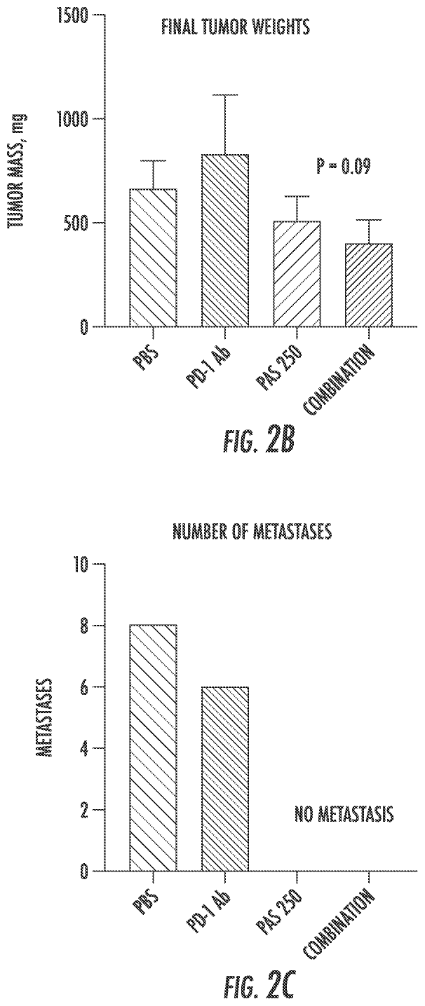

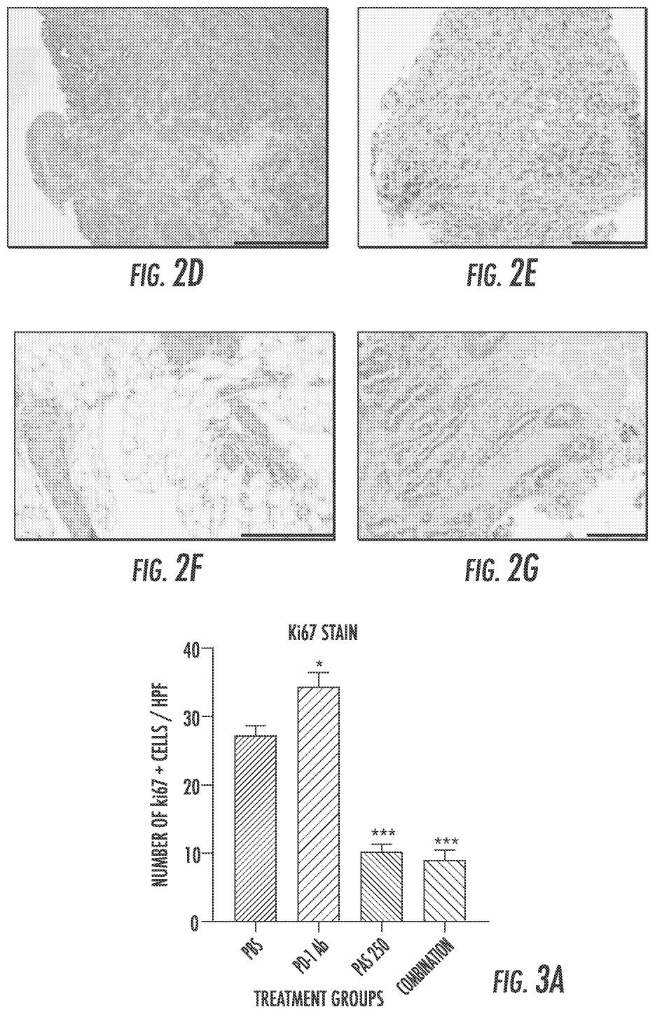

YTN gastric cancer tumor volumes measured over time are shown in FIG. 2A. Therapy with PD-1 Ab monotherapy had no effect on tumor growth compared to controls. In contrast, mice treated with PAS monotherapy or PAS in combination with PD-1 Ab had significantly slowed tumor growth over time. PAS monotherapy slowed tumor growth by 31% compared to PBS-treated controls (p=0.023). When PAS was given in combination with the PD-1 Ab the tumor growth was slowed by 59% compared to tumors of PBS-treated controls (P=0.0003). When the growth rate of tumors from PAS-vaccinated mice was compared to that of the tumors of mice treated with the combination therapy, the difference was statistically significant (p=0.0018). These results suggested that the combination therapy was better than PAS monotherapy. The mass of the tumors when excised was less in the PAS- and combination-treated mice, but this difference did not reach significance (FIG. 2B). The total number of metastases in each group were counted at autopsy and confirmed by histology. FIG. 2C shows the remarkable finding that there were no metastases in the mice treated with patent applications monotherapy or PAS combined with the PD-1 Ab. Hematoxylin & eosin staining confirmed that the tissues dissected from control mice and PD-1 Ab treated mice were metastases. FIGS. 2D-2G show representative histology of YTN metastases from the stomach wall, mesentery, peritoneum, and abdominal wall, respectively.

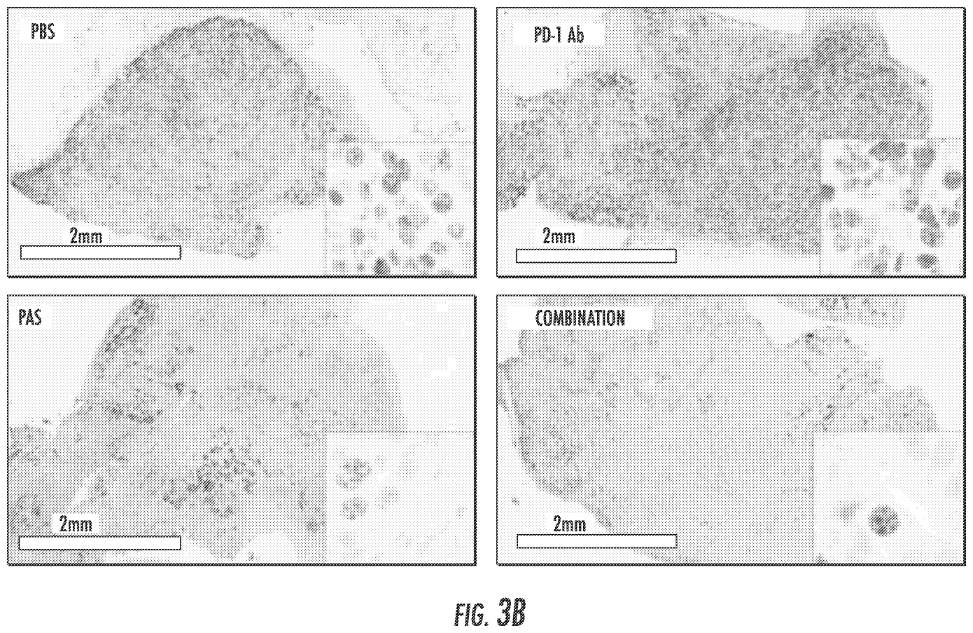

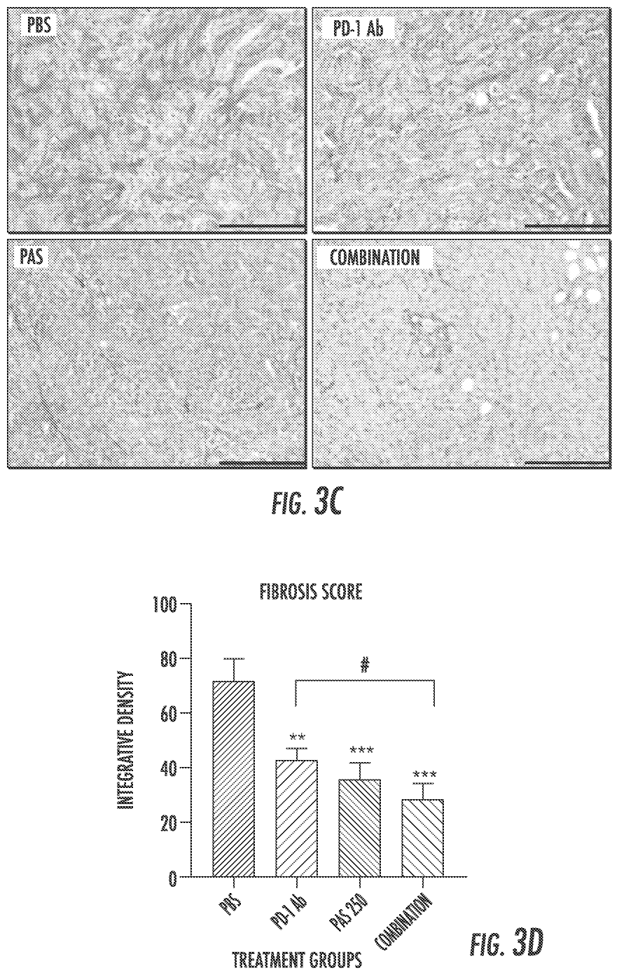

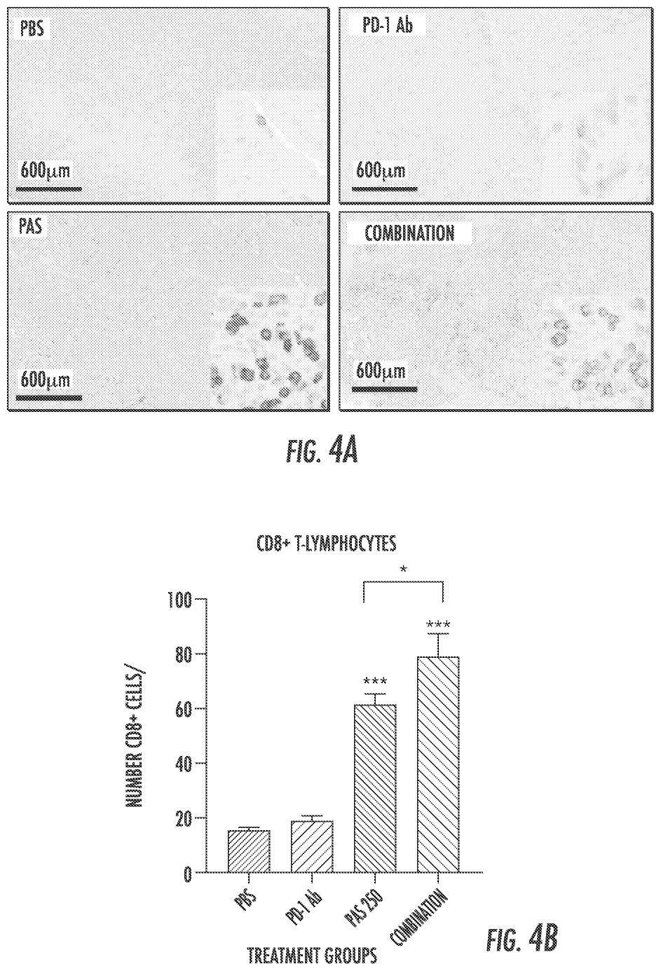

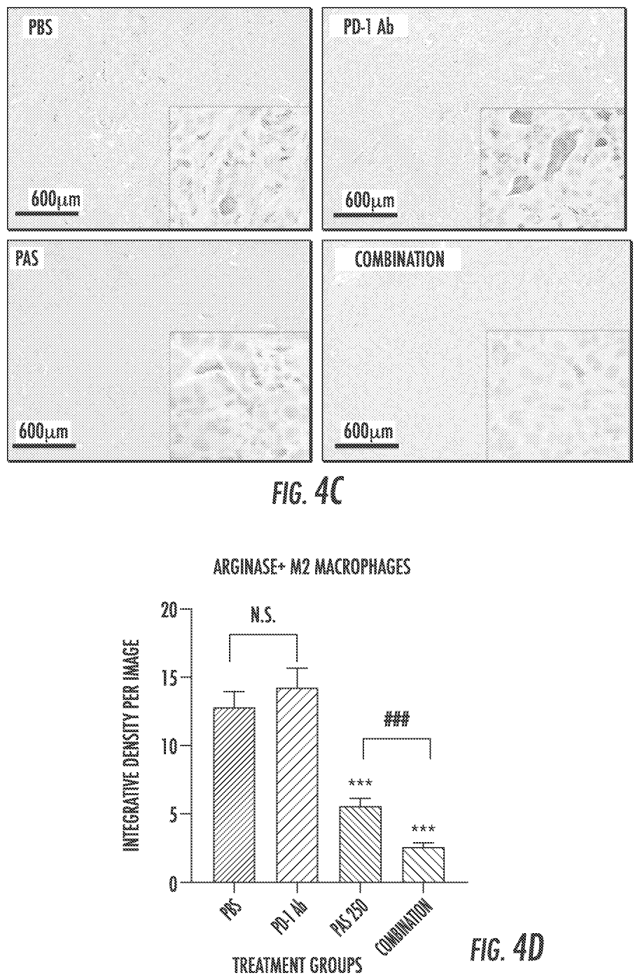

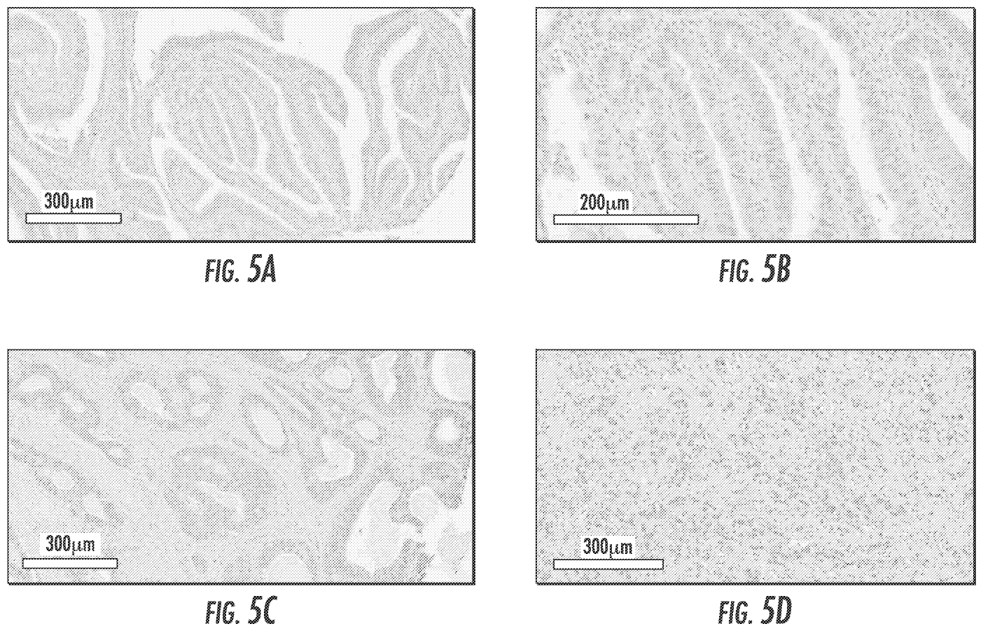

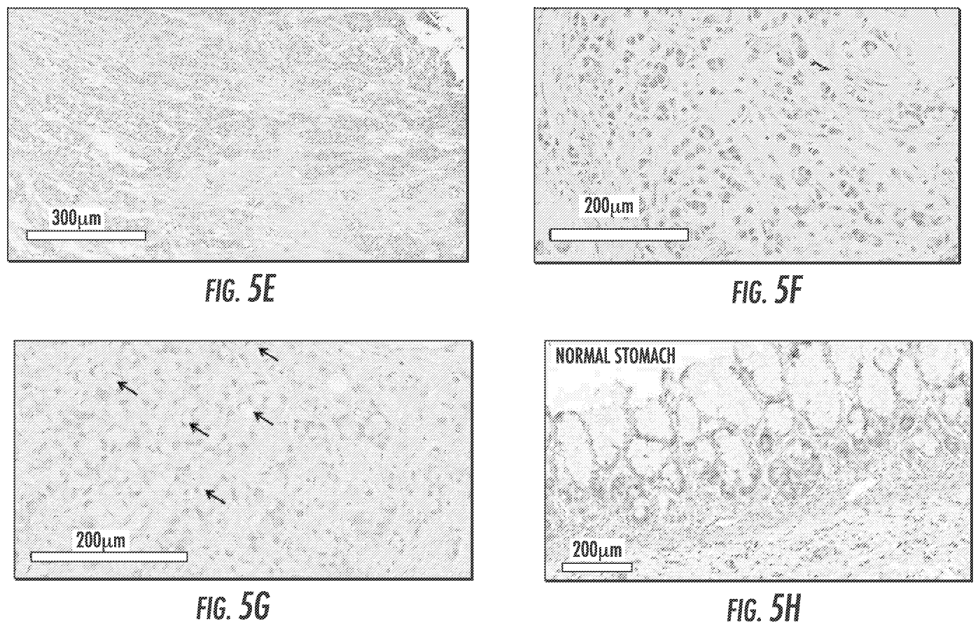

Another demonstration of the effects on tumor growth is the measurement of the Ki67 proliferation index. Ki67 immunoreactivity was increased in the tumors of PBS and PD-1 Ab treated mice (FIG. 3A). The proliferation index is significantly decreased in tumors of mice treated with PAS monotherapy or in combination with PD-1 Ab (FIG. 3A). A low power (magnification 2×) representative image from each treatment group is shown in FIG. 3B with a higher magnification (40×) insert image for each tumor. Marked Ki67 immunoreactivity is identified in tumors from PBS and PD-1 Ab treated mice. In contrast, the Ki67 staining is markedly decreased in tumors of mice treated with PAS with or without PD-1 Ab. These histologic sections confirm tumors of the PAS and combination-treated mice had decreased proliferation or growth rate.

### Example 4

### PAS and PD-1 Ab Combination Therapy Decreases Fibrosis in Gastric Cancer

Tumor fibrosis is thought to impede the penetration of chemotherapeutic agents into cancers and also restrict the influx of T-lymphocytes. YTN gastric tumors demonstrate characteristic dense fibrosis as seen in tumors of PBS-treated control mice with the Masson's trichrome stain of FIG. 3C. There is visibly less fibrosis noted in the tumors of mice treated with PAS monotherapy or PAS in combination with PD-1 Ab. Computerized analysis and quantification of the integrative density of fibrosis is shown for each treatment group in FIG. 3D. Although there was modest decrease in fibrosis in tumors of PD-1 Ab treated mice, when combined with PAS therapy, the amount of fibrosis was significantly further decreased.

### Example 5

### PAS and PD-1 Ab Combination Therapy Changes the Immune Cell Signature of Gastric Cancer

One reason for the lack of effect of immune checkpoint therapy in cancers is thought to be due to the lack of tumor infiltrating T-cells. Tumors from each treatment group were stained for CD8⁺ T-lymphocytes and the number of immunoreactive cells compared between groups. FIG. 4A shows the paucity of CD8⁺ T cells in gastric tumors of PBS control mice and in PD-1 Ab-treated mice. The number of CD8 immunoreactive cells is visibly increased in tumors of PAS-treated mice and mice treated with the combination therapy (FIG. 4A). Computer analysis of the YTN tumors stained with the CD8 antibody show marked increase in CD8⁺ T-lymphocytes in tumors of PAS-treated mice and even a significantly greater increase of CD8⁺ T-cells in mice treated with the combination therapy (FIG. 4B).

Tumors from each group also underwent immunohistochemical staining with an antibody for arginase to detect M2-polarized tumor-associated macrophages (TAMs). These immunosuppressive TAMs are abundant in the tumors of control mice and PD-1 Ab-treated mice (FIG. 4C). In contrast, there are noticeably fewer arginase⁺ TAMS in the gastric tumors of mice treated with PAS and the combination therapy. Computer analysis with integrative density of the images (FIG. 4D) confirms that the immunoreactivity is significantly decreased in tumors of PAS-treated mice. Tumors of mice treated with both PAS and the PD-1 Ab have even further decrease immunoreactivity of arginase positive TAMs (FIG. 4D).

### Example 6

### Human Gastric Cancer Expresses CCK-BR by Immunohistochemistry

Human gastric cancer epithelial cells were positive for CCK-BR immunoreactivity (FIGS. 5A-5H) implying that the administration of PAS to human subjects would also decrease that activation of this receptor by neutralizing gastrin. The most common histologic classification was described in Lauren, 1965, where cancers were categorized histologically into one of two types: intestinal or diffuse. FIGS. 5A-5C shows CCK-BR immunoreactivity in tissues from the human gastric cancer array with the intestinal-type histology showing the characteristic glands or tubules lines by epithelial cells. Histological diffuse gastric carcinoma cells lack cohesion and invade tissues independently or in small clusters (Correa, 2013). Representative diffuse type gastric cancers also expressed CCK-BR expression and are shown in FIGS. 5D and 5E. Mucinous gastric cancer (FIG. 5F) and signet ring gastric cancer (FIG. 5G) are less common histologic types of gastric cancer. Characteristic staining of CCK-BR positive cells in the glands of the normal human stomach are seen in FIG. 5H. There was not significant difference in the intensity of the CCK-BR staining according to the integrative density analyzed with ImageJ between tumors classified as Grade 1 (164.8±1.4), Grade 2 (159.7±1.4), and Grade 3 (162.8±1.1).

### Discussion of the EXAMPLES

Disclosed herein is evidence using two murine gastric cancer cells and a human tissue microarray that the gastrin:CCK-BR signaling pathway is important in stimulating growth of gastric cancer. CCK-BRs were expressed in both cell lines and exogenous gastrin stimulated cell growth in vitro confirming gastrin sensitivity. Immunocytochemistry revealed endogenous gastrin expression within the gastric cancer cells suggesting that gastric cancer may regulate its own growth by an autocrine mechanism. Since exogenously administered gastrin or endogenously produced gastrin from the cancer cells can activate the CCK-BR receptor resulting in cellular or tumor proliferation, strategies to interrupt the interaction of gastrin should inhibit growth. Indeed, it is shown that a vaccine that targets gastrin can inhibit growth of gastric cancer in mice and prevent metastases. The PAS vaccine when administered as monotherapy decreased tumor growth in mice; however, the tumor inhibitory effect was significantly affected by co-administration of the PD-1 Ab with PAS. A clear advantage of having a therapy such as the PAS vaccine that shows efficacy with monotherapy is that when treating subjects with gastric cancer, not all subjects are eligible for immune checkpoint antibody treatment or some may have experienced adverse effects from the immune checkpoint therapy; hence, monotherapy may provide an alternative option to treat these subjects. However, in those subjects eligible for immune checkpoint therapy, the addition of patent applications could significantly decrease tumor growth and prevent metastases. This vaccine, PAS, significantly decreased gastric cancer proliferation and this change was confirmed histologically with marked decreased in the number of Ki67 immunoreactive tumor cells. PAS therapy also decreased fibrosis in the tumor microenvironment. Vaccination with PAS also altered the tumor immune cell signature by increasing the number of CD8⁺ T-cells and decreasing the number of M2-polarized immunosuppressive macrophages rendering the tumor microenvironment more susceptible to other treatments, such as PD-1 Ab therapy.

Although the cancer cells expressed receptors for PD-L1, monotherapy with a PD-1 Ab did not significantly decrease gastric cancer growth or metastases. However, when PD-1 Ab therapy was administered in combination with PAS, there was a greater effect on tumor growth rate than with PAS therapy alone. One explanation for the enhanced effect of PAS with the PD-1 Ab cold be attributed to the marked increase in CD8⁺ T-cells when the two immune therapies are given together. Another beneficial finding of combined administration included the additive effect seen on the number of arginase positive M2-polarized macrophages. We previously described an additive effect on tumor inhibition in pancreatic cancer when PD-1 Ab therapy alone had no tumor inhibitory effects but when combined with PAS, the PD-1 Ab had a greater effect than PAS alone (Osborne et al., 2019a). In the prior study in pancreatic tumors, we also showed that PAS in combination with the PD-1 Ab decreased fibrosis in the tumor microenvironment. The decrease in fibrosis would be expected to facilitate the influx of T-cells.

Although certain experiments disclosed herein were performed in immune competent mice with syngeneic murine tumors, the results of the CCK-BR immunoreactivity on the human gastric cancer array support the important translational and clinical relevance of this work. It was determined that both murine gastric cancers (YTN and NCC) expressed CCK-BRs and when YTN tumor bearing mice were treated with a gastrin vaccine, the tumor growth and metastases significantly decreased. Since gastrin is the major ligand activating the CCK-BR and because PAS therapy induces neutralizing gastrin antibodies and gastrin-activated memory T-cells (Osborne et al., 2019a), the ability to decrease signaling at this receptor is central to inhibiting cancer growth. Sheng et al., 2020 demonstrated that that mature enterochromaffin-like cells (ECL) cells in the gastric corpus express CCK-BRs, and that that gastric isthmal progenitor cells also expressed CCK-BRs that responded to hypergastrinemia by supplying new ECL cells. Their elegant work supports the importance of gastrin as a trophic peptide activating the CCK-BR in the gastric mucosa. We previously showed that CCK-BRs are expressed on several human gastric cancer cell lines (Smith et al., 1998) and that gastrin-stimulated growth in vitro was only blocked by the selective CCK-BR antagonist, L265,260. The human gastric cancer tissue array immunoreactivity for the CCK-BR in numerous human gastric cancers in this current study suggests the importance of this receptor as a potential target for therapy in human subjects. The finding of CCK-BR staining in both the intestinal and diffuse histologic gastric cancer types suggests the broad implication of utilizing a therapy that targets this proliferative pathway. Although mucinous and signet ring histologic types occur less often, the prognosis with these histologic types is typically more severe (Taghavi et al., 2012). Tissues in the human gastric cancer array with these less frequent histologic types also stained positive for the CCK-BR suggesting the potential broad application of PAS therapy in gastric cancer.

Research on gastrin as an immunogen was initiated by Dr. Susan Watson in the early 1990's (Watson et al., 1996; Watson et al., 1999a; Watson et al., 1999b). Although not popular at the time, Dr. Watson decided to take an immune approach to treating GI cancers by producing high-affinity anti-G17 antibodies that could neutralize serum gastrin and cell-associated gastrin. Since it had previously been reported that serum gastrin levels are elevated in colorectal tumors (Smith et al., 1989), she decided to begin her investigation in that tumor (Watson et al., 1995; Watson et al., 1996). A wealth of clinical data was generated over the years that honed in on an appropriate adjuvant, dosing schedule, dose concentration and boosters required to produce high affinity anti-G17 antibodies. It was discovered that as antibody titers rose, serum gastrin levels decreased (Rocha-Lima et al., 2014). It was found that antibody titers could be followed and patients with titers 1.2 units above baseline on average doubled their survival times in colon, pancreatic and gastric cancers. There were some surprising results along the way. First, PAS was synergistic with Gemcitabine; and second, unexpected long-term survivors were observed in several studies including pancreatic cancer. These results lead to discussions that perhaps something more than neutralizing gastrin was occurring; however, the tools available today were not available then. Although there were positive studies in all three indications and a well characterized safety profile of the product, the development of PAS took a major set-back when the company funding its development failed. The last patients treated with PAS were in 2004.

In the last two years, a great deal has been learned about the mechanism of action of PAS. Not only does it produce high affinity anti-G17 antibodies, but PAS activates a cellular immunity response with increased memory T-cells, NKT-cells and gamma-delta cells (Osborne et al., 2019a). Furthermore, it consistently changed the microenvironment in several animal models (pancreatic and gastric) leading to a synergistic effect with checkpoint inhibitors (Osborne et al., 2019a). The prevention of metastases in mice treated with PAS (Osborne et al., 2019b) was due to the inhibition of epithelial mesenchymal transition, and this mechanism of action may help explain the long-term survivors previously observed in the clinical program. The decreased fibrosis with patent applications therapy observed may help to explain the synergy previously found with gemcitabine. PAS vaccination in a precancerous KRAS murine model demonstrated that PAS not only decreases pancreatic fibrosis and alters the immune cell signature of the tumor microenvironment but that it also decreases proliferation and progression on precancerous PanIN lesions preventing pancreatic cancer (Smith et al., 2021). The data are compelling for its return to the clinic with a much better understanding of how to use the product.

### REFERENCES

All references listed in the instant disclosure, including but not limited to all patents, patent applications and publications thereof, scientific journal articles, and database entries (including but not limited to GENBANK® biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.
Adams et al. (1993) Cancer Res 53:4026-4034.
Alt et al. (1999) FEB S Lett 454:90-94.
Berkow et al. (1997) The Merck Manual of Medical Information, Home ed., Merck Research Laboratories, Whitehouse Station, New Jersey, United States of America.
Bird et al. (1988) Science 242:423-426.
Clackson et al. (1991) Nature 352:624-628.
Coloma et al. (1997) Nature Biotechnol 15:159-163.
Correa (2013) Gastroenterol Clin North Am 42:211-217.
Duch et al. (1998) Toxicol Lett 100-101:255-263.
Ebadi (1998) CRC Desk Reference of Clinical Pharmacology. CRC Press, Boca Raton, Florida, United States of America.
Freireich et al. (1966) Cancer Chemother Rep 50:219-244.
GENBANK® Accession Nos. AAA52771.1; NM_000805.5; NP_000725.1; NP_000796.1; NP_001002026.1; NP_001139345.1; NP_001158089.1; NP_001106862.1; NP_001230007.1; NP_001243.1; NP_005663.2; NP_005814.2; NP_006130.1; NP_031668.3; NP_741969.1; and Q13982-1.
Gerbino (2005) Remington: The Science and Practice of Pharmacy. 21st Edition. Lippincott Williams & Wilkins, Philadelphia, Pennsylvania, United States of America.
Glockshuber et al. (1990) Biochemistry 29:1362-1367.
Goodman et al. (1996) Goodman & Gilman's the Pharmacological Basis of Therapeutics, 9th ed., McGraw-Hill Health Professions Division, New York, New York, United
States of America.
Halland et al. (2020) J Clin Invest 130:2673-2688.
Holliger et al. (1993) Proc Natl Acad Sci USA 90:6444-6448.
Hu et al. (1996) Cancer Res 56:3055-3061.
Huston et al. (1988) Proc Natl Acad Sci USA 85:5879-5883.
Huston et al. (1993) Int Rev Immunol 10:195-217.
Katzung (2001) Basic & Clinical Pharmacology, 8th ed., Lange Medical Books/McGraw-Hill Medical Pub. Division, New York, New York, United States of America.
Kipriyanov et al. (1995) Cell Biophys 26:187-204.
Kipriyanov et al. (1998) Int J Cancer 77:763-772.
Kipriyanov et al. (1999) J Mol Biol 293:41-56.
Kohler et al. (1975) Nature 256:495.
Kontermann et al. (1999) J Immunol Meth 226:179-188.
Kortt et al. (1997) Protein Eng 10:423-433.
Kostelny et al. (1992), J Immunol 148:1547-1553.
Kurucz et al. (1995) J Immunol 154:4576-4582.
Lauren (1965) Acta Pathol Microbiol Scand 64:31-49.
Le Gall et al. (1999) FEBS Lett 453:164-168.
Marks et al. (1991) J Mol Biol 222:581-597.
McCartney et al. (1995) Protein Eng 8:301-314.
Muller et al. (1998) FEBS Lett 432:45-49.
Neesse (2013) Onco Targets Ther 7:33-43.
Osborne et al. (2019a) Cancer Immunol Immunother 68:1635-1648.
Osborne et al. (2019b) Am J Physiol Gastrointest Liver Physiol 317:G682-G693.
Pack et al. (1992) Biochemistry 31:1579-1584.
Park et al. (2015) Mol Carcinog 54:1521-1527.
Patel et al. (1999) Gene Therapy 6:412-419.
PCT International Patent Application Publication Nos. WO 1992/22653, WO 93/25521, WO 2003/005955, WO 2005/095459, WO 2020/135674.
Pegram et al. (2012) Blood 119:4133-4141.
Rehfeld et al. (1989) Cancer Res 49:2840-2843.
Remington et al. (1975) Remington's Pharmaceutical Sciences, 15th ed., Mack Pub. Co., Easton, Pennsylvania, United States of America.
Rocha-Lima et al. (2014) Cancer Chemother Pharmacol 74:479-486.
Sadelain et al. (2009) Curr Opin Immunol 21:215-223.
Segal et al. (2014) J Clin Oncol 32(5 s):abstr 3002.
Shalaby et al. (1992) J Exp Med 175:217-225.
Sheng et al. (2020) Cell Mol Gastroenterol Hepatol 10:434-449.
Singh et al. (1986) Cancer Res 46:1612-1616.
Smith & Solomon (1988) Gastroenterology 95:1541-1548.
Smith et al. (1989) Dig Dis Sci 34:171-174.
Smith et al. (1990) Dig Dis Sci 35:1377-1384.
Smith et al. (1991) Regul Pept 32:341-349.
Smith et al. (1995) Am J Physiol 268:R135-R141.
Smith et al. (1996) Am J Physiol 271:R797-R805.
Smith et al. (1998) Int J Oncol 12:411-419.
Smith et al. (2021) Cancer Prey Res (Phila) 14(10):933-944.
Speight & Holford (eds.) (1997) Avery's Drug Treatment: A Guide to the Properties, Choice, Therapeutic Use and Economic Value of Drugs in Disease Management, 4th ed., Adis International, Philadelphia, Pennsylvania, United States of America. Stephan et al. (2007) Nat Med 13:1440-1449.
Taghavi et al. (2012) J Clin Oncol 30:3493-3498.
Templeton & Brentnall (2013) Surgical Clinics of North America 93(3):629-645.
U.S. Patent Application Publication Nos. 2006/0188558, 2007/0036773, 2009/0180989, 2009/0257991, 2011/0038836, 2012/0058051, 2012/0213783, 2012/0252742, 2017/0145095, 2017/0369561, 2018/0230224, 2018/0244796, 2019/0046659, and 2020/0140520.
U.S. Pat. Nos. 3,598,122; 4,816,567; 5,016,652; 5,234,933; 5,234,933; 5,326,902; 5,607,676; 5,609,870; 5,622,702; 5,785,970; 5,866,128; 5,935,975; 6,106,856; 6,162,459; 6,172,197; 6,180,082; 6,248,516; 6,291,158; 6,410,319; 6,495,605; 6,582,724; 6,861,510; 8,389,282; 8,802,374; 9,272,002; 9,988,452; 10,113,003; 10,183,992; 10,301,391; 10,358,492; and U.S. Pat. No. 10,377,822.
Upp et al. (1989) Cancer Res 49:488-492.
Watson et al. (1989) Br J Cancer 59:554-558.
Watson et al. (1995) Int J Cancer 61:233-240.
Watson et al. (1996) Cancer Res 56:880-885.
Watson et al. (1998) Int J Cancer 75:873-877.
Watson et al. (1999a) Int J Cancer 81:248-254.
Watson et al. (1999b) Gut 45:812-817.
Watson et al. (2006) Nature Reviews Cancer 6:936-946.
Whitlow et al. (1991) Methods Companion Methods Enzymol 2:97-105.
Yamamoto et al. (2018) Cancer Sci 109:1480-1492.
Zhu et al. (1997) Protein Sci 6:781-788.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A method for enhancing an anti-tumor and/or anti-cancer immunotherapy, the method comprising administering to a tumor and/or a cancer a composition comprising, consisting essentially of, or consisting of a conjugate comprising, consisting essentially of, or consisting of a gastrin immunogen conjugated to an immunogenic carrier, optionally conjugated via a linker, in an amount and via a route sufficient to enhance entry into the tumor and/or cancer of an anti-tumor T cell, whereby an anti- and/or anti-cancer tumor immunotherapy is enhanced.

2. The method of claim 1, wherein the tumor and/or the cancer is a gastrointestinal tumor and/or cancer, optionally a gastrin-dependent gastrointestinal tumor and/or cancer, further optionally a pancreatic tumor and/or cancer, and/or is a gastrin-responsive cancer, optionally a gastrinoma, lung cancer, and/or thyroid cancer.

3. The method of claim 1 or claim 2, wherein the tumor and/or the cancer is a solid tumor and/or cancer of the gastrointestinal tract, optionally a solid tumor and/or cancer of the pancreas.

4. The method of any one of claim 1-3, wherein the immunogenic carrier is selected from the group consisting of diphtheria toxoid, tetanus toxoid, keyhole limpet hemocyanin, and bovine serum albumin.

5. The method of any one of claims 1-4, wherein the linker comprises a ε-maleimido caproic acid N-hydroxysuccinamide ester.

6. The method of any one of claims 1-5, wherein the linker and the gastrin peptide are separated by an amino acid spacer, optionally wherein the amino acid spacer is between 1 and 10 amino acids in length, further optionally wherein the amino acid spacer is 7 amino acids in length.

7. The method of any one of claims 1-6, wherein the composition further comprises an adjuvant, optionally an oil-based adjuvant.

8. The method of any one of claims 1-7, wherein the gastrin peptide comprises, consists essentially of, or consists of an amino acid sequence selected from the group consisting of EGPWLEEEEE (SEQ ID NO: 1), EGPWLEEEE (SEQ ID NO: 2), EGPWLEEEEEAY (SEQ ID NO: 3), and EGPWLEEEEEAYGWMDF (SEQ ID NO: 4).

9. A pharmaceutical composition for use in producing a medicament for treating a gastrin-associated tumor and/or cancer and/or a gastrin-responsive tumor and/or cancer, the pharmaceutical composition comprising, consisting essentially of, or consisting of a conjugate comprising a gastrin immunogen in an amount sufficient to enhance anti-tumor and/or anti-cancer T cell entry into the gastrin-associated tumor and/or cancer.

10. A pharmaceutical composition for use in treating a tumor and/or a cancer, optionally, a gastrin-associated tumor and/or cancer, the pharmaceutical composition comprising, consisting essentially of, or consisting of a conjugate comprising a gastrin immunogen in an amount sufficient to enhance anti-tumor and/or anti-cancer T cell entry into the tumor and/or cancer.

11. A method for treating a tumor and/or cancer, optionally a gastrin-associated tumor and/or cancer and/or a gastrin-responsive tumor and/or cancer, in a subject, the method comprising:
(a) administering to the subject a first composition comprising, consisting essentially of, or consisting of a conjugate comprising, consisting essentially of, or consisting of a gastrin immunogen conjugated to an immunogenic carrier, optionally conjugated via a linker, in an amount and via a route sufficient to enhance anti-tumor and/or anti-cancer T cell entry into the tumor and/or cancer; and
(b) administering to the subject a second compositions comprising an anti-tumor and/or anti-cancer T cell, wherein the anti-tumor and/or anti-cancer T cell optionally comprises a chimeric antigen receptor (CAR) that binds to a tumor-associated and/or cancer-associated antigen present on the tumor and/or the cancer,
whereby the tumor and/or the cancer is treated.

12. The method of claim 11, wherein the tumor and/or the cancer is a gastrointestinal tumor and/or cancer, optionally a gastrin-dependent gastrointestinal tumor and/or cancer, further optionally a pancreatic tumor and/or cancer.

13. The method of claim 11 or claim 12, wherein the immunogenic carrier is selected from the group consisting of diphtheria toxoid, tetanus toxoid, keyhole limpet hemocyanin, and bovine serum albumin.

14. The method of any one of claims 11-13, wherein the linker comprises a ε-maleimido caproic acid N-hydroxysuccinamide ester.

15. The method of any one of claims 11-14, wherein the linker and the gastrin peptide are separated by an amino acid spacer, optionally wherein the amino acid spacer is between 1 and 10 amino acids in length, further optionally wherein the amino acid spacer is 7 amino acids in length.

16. The method of any one of claims 11-15, wherein the composition further comprises an adjuvant, optionally an oil-based adjuvant.

17. The method of any one of claims 11-16, wherein the gastrin peptide comprises, consists essentially of, or consists of an amino acid sequence selected from the group consisting of EGPWLEEEEE (SEQ ID NO: 1), EGPWLEEEE (SEQ ID NO: 2), EGPWLEEEEEAY (SEQ ID NO: 3), and EGPWLEEEEEAYGWMDF (SEQ ID NO: 4).

18. The method of any one of claims 11-17, wherein the CAR binds to an antigen selected from the group consisting of a claudin18.2 antigen, a glypican3 antigen, a mesothelin antigen, a carcinoembryonic antigen (CEA), a prostate stem cell antigen (PSCA), and a CD70 antigen.

19. A method for sensitizing a solid tumor and/or cancer in a subject to a chimeric antigen receptor-T (CAR-T) cell therapy, the method comprising:
(a) administering to the subject a first composition comprising, consisting essentially of, or consisting of a conjugate comprising, consisting essentially of, or consisting of a gastrin immunogen conjugated to an immunogenic carrier, optionally conjugated via a linker, in an amount and via a route sufficient to enhance entry of the CAR-T cell into the solid tumor and/or cancer; and
(b) administering to the subject a second composition comprising, consisting essentially of, or consisting of a CAR-T cell that is targeted against an antigen present within the solid tumor and/or cancer,
whereby the solid tumor and/or cancer in the subject is sensitized to the CAR-T cell therapy.

20. The method of claim 19, wherein the solid tumor and/or the cancer is a solid gastrointestinal tumor and/or cancer, optionally a solid gastrin-dependent gastrointestinal tumor and/or cancer, further optionally a solid pancreatic tumor and/or cancer.

21. The method of claim 19 or claim 20, wherein the immunogenic carrier is selected from the group consisting of diphtheria toxoid, tetanus toxoid, keyhole limpet hemocyanin, and bovine serum albumin.

22. The method of any one of claims 19-21, wherein the linker comprises a ε-maleimido caproic acid N-hydroxysuccinamide ester.

23. The method of any one of claims 19-22, wherein the linker and the gastrin peptide are separated by an amino acid spacer, optionally wherein the amino acid spacer is between 1 and 10 amino acids in length, further optionally wherein the amino acid spacer is 7 amino acids in length.

24. The method of any one of claims 19-23, wherein the composition further comprises an adjuvant, optionally an oil-based adjuvant.

25. The method of any one of claims 19-24, wherein the gastrin peptide comprises, consists essentially of, or consists of an amino acid sequence selected from the group consisting of EGPWLEEEEE (SEQ ID NO: 1), EGPWLEEEE (SEQ ID NO: 2), EGPWLEEEEEAY (SEQ ID NO: 3), and EGPWLEEEEEAYGWMDF (SEQ ID NO: 4).

26. The method of any one of claims 19-25, further comprising administering to the subject one or more additional anti-tumor and/or anti-cancer therapies.

27. The method of claim 26, wherein the one or more additional anti-tumor and/or anti-cancer therapies comprises, consists essentially of, or consists of administering to the subject an immune checkpoint inhibitor.

28. The method of claim 27, wherein the immune checkpoint inhibitor inhibits a biological activity of a target polypeptide selected from the group consisting of cytotoxic T-lymphocyte antigen 4 (CTLA4), programmed cell death-1 receptor (PD-1), and programmed cell death 1 receptor ligand (PD-L1).

29. The method of claim 27 or claim 28, wherein the immune checkpoint inhibitor is selected from the group consisting of Ipilimumab, Tremelimumab, Nivolumab, Pidilizumab, Pembrolizumab, AMP514, AUNP12, BMS-936559/MDX-1105, Atezolizumab, MPDL3280A, RG7446, R05541267, MEDI4736, and Avelumab.

30. The method of any one of claims 26-29, wherein the method reduces and/or inhibits growth of the tumor and/or the cancer in the subject.

31. The method of any one of claims 26-30, wherein the composition is administered in a dose selected from the group consisting of about 50 μg to about 1000 μg, about 50 μg to about 500 μg, about 100 μg to about 1000 μg, about 200 μg to about 1000 μg, and about 250 μg to about 500 μg, and optionally wherein the dose is repeated once, twice, or three times, optionally wherein the second dose is administered 1 week after the first dose and the third dose, if administered, is administered 1 or 2 weeks after the second dose.

32. The method of any one of claims 26-31, wherein the one or more additional anti-tumor and/or anti-cancer therapies is administered subsequent to the administration of at least the first dose of the composition.

33. The method of any one of claims 11-17, wherein the CAR binds to an antigen selected from the group consisting of a claudin18.2 antigen, a glypican3 antigen, a mesothelin antigen, a carcinoembryonic antigen (CEA), a prostate stem cell antigen (PSCA), and a CD70 antigen.
